SEQUENCE LISTING (7 sequences)

SEQ ID NO: 1 - DNA, 4961 bp, Saccharum spontaneum
  source 1..4961: mol_type = genomic DNA
ttaattaagctagccttgagctttgcgctggcagagcggctcagagtggtattgctgccaccggtgcgtgcagactcgtcccgaaggtcaccggagggataaccggttgcgtcacctccatggcctgtgggagaggcctcaccgtggctatggttacacaccgagaagccgagtccaagttagagtcaatgattgaacaacaaaaggaagaaaattttaaggaacaaggctaacccggtagtggagtgcctcatccagctaggatggcgtgcctacctgagggttggactcggctgatgggtgggtccgagcctatgcgaaggggccccagagcggatggagtactaggttccctttgggagcttctcgcgatgtcgctcggtgagctcctcggtcggcatcttagcggcacgcttgtcatcaacctggggaagtgcgggagtgttggggggagagagacatacttctcatgaacctgtcatagcaaatttggagaaatcaagaaatcaagacttgaatgaagcaaatacagagattaaccactcagatcttatctttgaacagggtcggcggaggttgtacatgccagggccgttcttgtactcagccaccagctctgcagggatctgactggcctcaaagaattcatcgaccagtctctccaactctgcttccaggaggttctctgtggactccctcgttggctcttcaactcccgcaaagtcaaactctggatgcactcggctcttgatgggagcgatgcgtggatcaggcaactcactaaaatgctcgtaggcgtgagtcctgccttcttctccttcaggatcttgatgagatttggcacttgggccgactcgacagcagaaggaagaagggtccaataaccatgaggagttggagactggctagaaaagggggagggaaccgtcaatgttcttgacgtaaaacgactcggtgttctagcttttgttcgagctcttccaaataagtgcttttttaacctctcacggagctggaagccagctccccccacaagattggggctcacccaagtggggtggggcttaagggtgaaaagaaattgaaagaaagccaagtgtggcagaatgcccaagaatgctttgcaaatgtggataaaagcacaaatgtgcgaaatggagtttgggttgaggtgatgagcctcgagcccatagtaatcaagaaggccacgaaagaagggagagattgggatctcgaatccacgtgggcaaaaggactcgaggtgtagtacctcgtttgtgtctggcgtggggacaacctctgcttccgcgggccgccagttaacaacctccttcaccggcaaatggtcacgctcgaccaatgtcctgagtctcccatcggagctcacagacatcggccatgtcctgtcctccttggatgtcgccttcgaagccttcaccaccttcttgctgcgacccatggtccgattggtggaggtggttgaatggggcggagagggttttggttcaagagagagaaagagagatcacaagagctcaagaggcgtgggcgcaaggaggaagagaagagcaaatggctgatatgctggcgtaatggacagaagggggccatttggggggatataaaggtgtgactacacgccccctcagctaccccgtcgccgttaatgacggagtatgccaaccgatcgttcacattagggttttagccttagtgggctttgatggaacgccacctcgacataatgaggcgtgtgaatcggggcgtggctacaatatttactaacatttatcagatcctagttgtcccaatatccgagaaaaagatttttgtcttactcgagaaggaacccaagactgctagctttttataagcattcgagtgaccacgactcagcccggtggcacaccttttgaggttgaggtacatgccactaccactagtaggatgatggtgccactcgggaacctctcaattgggaagttggtatgctcttgggagccgaatcactcgcttgataggtgtgactcggataccttacgaacaactcggctagagggtacgtacgatgtgctgatgactcggttctttaataaaagacgaggtgccctctaaagccttttatgggtcggattgtgagtccttcccaatcactctgccgacattaaaattgaccatgcgagattttttagcctacgagagcactcagctggtaagatgggttagcaacatcgggattgcgaaggactcaggggctgctgatgatgacacataacccgggtatctactagtgggcttaggccaccactcggataggggtgggccgggccgccaggtcaagaccagctgctatacgactcggaataacactgccatgtgccccaaggtgtcggaagtccaagattagctaggcttttataaacctgttgtaatacgatacgaaagataggtgccgacttggctcaggactccgattgtaatcctgcctcttagactatataactaggggcagggtacccccttcaaacattcaatctgtctccagcataagaaaacaatagccaccaaagcaggacatagggtattacgccattggcggcctgaacctgtctaaatcgtgtctctatgattttcctcgacaaccctacatataaatctactcgtctgcacaaagctatgacaactatatattgtaaaattactttcatgaagccccaagtaacataaactttgtattgtcaatatatgtaactatttatatatcgatgatattcacatgtaactaattttatggtcaatgttaaaaaaaattgaatcagtaaacaacttatatttcagattggatggagtatgaaacagtggttgaaaatatctcaagcactgattgaagcatgacgacaaagcttccgattgcaataatgagaacatataggtggcatctcggctttctacgtggactaatgacaaagagaagatgcacatcatgccgcatataattcaatggactaatgatggtttatcggctcaagcattaacaatagacatcagtggattgggaaggcagacccagcatcttctttcatgagatagaacacgcaatgtgacccaaagtaagtatattctctctcgatttcttcctttcattccaggtccaaccaaatcatgatgttgtaggatgatgtgttacaccatcagaacaagaagatctttctttttttttctcgagcgaggttagagcacatacaaggtataatatcagctgcctgtaagagttgccacgtaggatttctagcgatgcaaagaggttataatgaggagagagattgttgtctcttgacgaaatgactatctataatataagaaagagtcgagatgtgtcttgtaggatatctactatgaggcaacaacaatagattgtgggttatacaagctgtcatttgaattgtctaaaaatgacatgtacagtgtatagacagcagccatttcaactgttgtatgtatccttacagaatgatagaagtaaaactctcatgcaccaaatgcttgacaagggctctagagtttacgcggtatgcaggcctagactcaactcataagctaccctatttggtcttgccatgtaaattaaatacggtaattattatcttgggatacatgtcagaatatgatgtaattattacttgttcaaaagaatatcttcaatttggcattgtctttttcgttcgaactaactctgactgttaatgactatcaacacatactgacaggatgtacctatctttcttgatcggaagagatggacagttaaccttgaatatatttggacgacacatacattgcattattctttttgacggctgggagaagccgtaccctaccgaatagccatgaaatatgttaggttcctaggacggttttaaaattctaatgtcgttttggctgtccctcaataaacctagctactcggtgataatctgaaagcgttacattgacacatgaataagacagcacaccacattgcattattctttttgacggctgggagaagccataccataccgaatagccatgaaatatgtcagattcgtagcacggttttaaaattctaatatcgttttggctgtccctcaataaacccagctactcggtgacattctgaaagcattacattgacacatgaatgagacagcacaccacattgcattattctttttgacggctgggagaagtcataccataccgaatagccatgaaatatgttagattcctagcacggttttaaaattctaatactccctccgttcatttatctccatcattttactcttcggcgtagtgaccaaggagcagagtaaaaccactcagtttgcatttaatcatcgcgtgctgagcgtatacgaggagtcacaggttaggaacgcagtaaagagacaataaatacaaacgggtggtagccaatgagcagcaaacagaaaataaatgcgggcaagtcccaagctaatatgaagaagatttttaaataaaaagtttttgcttagatgaaggagataaataaatggagggaatatcgttttagctgtccctcaataaacccaactactcggtgacaatccgaaagcgttatattgacacatgactgagacagcacaccatcctctttcccctatcgcttggctccatctccatcacttcccaggggccaggggccaaataggagcgggcaaataatggacaaaaattacttctttaatcgaatgcccaccctgccaaattccaaggattccttcctccagatataagagtcatcgtccatgggtttcttggccgcagctccttcagttggctcttggtcggcttcttgcccattactgctcttcctgagagaacctcgaattccgcaagaatttaactcgctgttgcagggaccgttgagctcacgtgcagggggaaaacgcggccgc

SEQ ID NO: 2 - DNA, 4961 bp, Saccharum spontaneum
  source 1..4961: mol_type = genomic DNA
ttaattaaaaggatggcactgaagcataaaaattaagacttcaagattaagtaggagagagctctacttacctgtcgaagcggcgctggaaggtgggggacaccactcggagtgctagccttgagctttgcgctggcagagcagctcagagtggtattgctgccaccggtgcgtgcagactcgtcccgaaggtcaccggagggataacggttgcgtcacctccatggcctgtgggagaggcctcaccgtggctatggttacacaccgagaagccgagtccaagttagagtcaatgattgaacaacaaaaggaagaaaattttgcctaacccggtagtggaggatccagctgagggttggacctgagggtccgagcctgatggagtactaggtccctttgggagcttctcgtgctgtcgctcggtgagctcctcggtcggcgtcttagcggcacgcttgtcatcaacctggggaagtgcgggagtgttggggggagagagacatacttctcatgaacctgtcacagcaaatttggagaaatcaagaaatcaagactcgaatgaagcaaatacagagattaaccactcagatcttatctttgaacagggtcggcggaggttgtacatgctagggccgttcttgtactcagccaccagctctgcagggatctgactggcctcaaagaattcgtcgaccagtctctccaactctgcttctaggaggttctctgtggactccctcgttggctcttcaactcccgcaaagtcaaactctggatgcactcggctcttgatgggagcgatgcgtggatcaggcaactcactaaaatgctcgtaggcatgagtcctgccttcttctcctttaggatcttgatgagatttggcacttgggccgactcgacagcagaaggaagaagggtccagtaaccatgaggagttggagactggctagaaaagggggagggaaccgtcaatgttcttgacgtaaaacgactcggtgttctagcttttgttcgaggtcttgcaaataagtgcttttttaacctctcacagagctggaagccagctcccccacaagattggggctcacccaagtggggtggggcttaagggtgaaaagaaattgaaagaaagccaagtgtggcagaatgcccaagaacgcttcgcaaatgtggataaaaagcacaaatgtgcgaaatggagtttgggttgaggtgatgagcctcgagcccatagtaatcaagaaggccacgaaagaagggagagattgggatctcgaatccacgtgggcaaaaggactcgaggtgtagtacctcgtttgtgtctggcgtggggacaacctctgcttccgcgggccgccagttaacaacctccttcaccggcaaatggtcacgctcgaccaatgtcctgagtctcccatcggagctcacagacatcggccatgtcctgtcctcctcagatgtcgcctttgaagccttcaccaccttcttgctgcgacccatggtccgattggtggaggtggttgaatggggtggagagggttttggttcaagagagagaaagagagaagatcacaagagctcaagaggcgtgggcgcaaggaggaagagaagagcaaatggctgatatgctggcgtaatggacagaagggggccatttggggggatataaaggcgtgactacacgccccctcagctcccccgtcgccgttaatgacggagtatgccaaccgatcgttcacattagggttttagccttagtgggctttgatggaacgccacctcgacataatgaggcgtgtgaatcggggcgtggctacagtatttactaacatttatcagatcctagttgtcccaatatccgagaagcattcgagtgaccacgactcagcccggtggcacaccttttgaggttgaggtacatgccactaccactagtaggatgatggtgccactcgggaacctctcaattgggaagctggtatgctctcgggagccgaatcactcgcttgataggtgtgactcggataccttacgaacaactcggctatagggtacgtacgatgtgctgatgactcggttctttaataaaagacgaggtgccctctaaagccttttatgggtcggattgtgagtccttcccaatcactctgccgacattaaaattgaccatgcgagattttttagcctacgagagcactcagctggtaagatgggttagcaacatcgggattgcgaaggactcaggggctgctgctgatgacacataacccgggtatctactagatgggcttaggccaccactcggatagggggtgggccaggccgccaggtcaagaccagctgctatacgactcggaataacactgccatgtgccccaaggtgtcggaagtccaagattagctaggctttctataaacctgttgtaatacgatacgaaagataggtgccgacttggctcaggactccgattgtaatcctgcctcttagactatataactaggggcagggtacccccttcaaacattcaatctcgtctccagcataagaaaacaatagccaccaaagcaggacatagggtattacgccattggcggcctgaacctgtctaaatcgtgtctctatgattttcctcgacaaccctacatataaatctactcgtctgcacaaagctatgacaactatatattgtaaaattactttcatgaagccccaagtaacataaactttgtattgtcaatatatgtaactatttatatatcgatgatattcacatgtaactaattttatggtcaatgttaaaaaaaattgaatcagtaaacaacttatatttcagattggatggagtatgaaacagtggttgaaaatatctcaagcactgattgaagcatgacgacaaagcttccgattgcaataatgagaacatataggtggcatctcggctttctacgtggactaatgacaaagagaagatgcacatcatgccgcatataattcaatggactaatgatggtttatcggctcaagcattaacaatagacatcagtggattgggaaggcagacccagcatcttctttcatgagatagaacacgcaatggtgacccaaagtaagtatattctctctcgatttcttcctttcattccaggtccaaccaaatcatgatgttgtaggatgatgtgttacaccatcagaacaagaagatctttctttttttttttctcgagcgaggttagagcacatacaaggtataatatcagctgcctgtaagagttgccacgtaggatttctagcgatgcaaagaggttataatgaggagagagattgttgtctcttgacgaaatgactatctataatataagaaacgagtcgagatgtgtcttgtaggatatctactatgaggcaacaacaatagattgtgggttatacaagctgtcatttgaattgtctaaaaatgacatgtacagtgtatagacagcagccatttcaactgttgtatgtatccttacagaatgatagaagtaaaactctcatgcaacaaatgcttgacaagggctctagagtttacgcggtgtgcatgcctagactcaactcataagctaccctatttggtcttgccatgtaaattaaatacggtaattattatcttgggatacatgtcagaatatgatgtaattattacttgttcaaaagaatatcttcaatttggcattgtctttttcgttcgaactaactctgactgttaatgactatcaatacatactgacaggatgtacctatctttcttgatcggaagagatggacagttaaccttgaatatatttggacgacacatacattgcattattctttttgacggttgggagaagccgtaccataccgaatagccatgaaatatgttaggttcctaggacggttttaaaattctaatgtcgttttggatgtccctcaataaacccagctactcggtgataatctgaaagcgttacattgacacatgaataagacagcacaccacattgcattattctttttgacggctgggagaagccataccataccgaatagccatgaaatatgtcagattcgtagcacggttttaaaattctaatatcgttttggctgtccctcaataaacccagctactcggtgacattctgaaagcattacattgacacatgaatgagacagcacaccacattgcattattctttttgacggctgggagaagccataccctaccgaatagccataaaatatgttagattcctagcacggttttaaaattctaatactccctccgttcatttatctccatcattttagccttcggcgtagtgaccaaggagcagagtaaaaccacttagtttgcatttaatcatcgcgtgctgagcgcatacgaggagtcacaggttaggaacgcagtaaagagacaataaatgcaaatgagtggtagccaatgagcagcaaacagaaaataaatgcgggcaagtcccaagctaatatgaaggagatttttgaataaaaagtttttgcttagatgaaggagataaatgaatggagagaatatcgttttagctgtccctcaataaacccaactactcggtgacaatccgaaagcgttatattgacacatgactgagacagcacaccatcctctttcccctatcgcttggctccatctccatcacttcccaggggccaggggccaaataggagcgggcaaataatggacaaaaattacttctttaatcgaatgcccaccctgccaaattccaaggattccttcctccagatataagagtcatcgtccatgggtttcttggccgcagctccttcagttggctcttggtcggcttcttgcccattactgctcttcctgagagaacctcgaattccgcaagaatttaactcgctgttgcagggatcattgagctcacgtgcagggggaaaacgcggccgc

SEQ ID NO: 3 - DNA, 4934 bp, synthetic construct
  source 1..4934: mol_type = other DNA
ttaattaacggctcagagtggtattgctgccaccggtgcgtgcagactcgtcccgaaggtcaccggagggataaccggttgcgtcgcctccatggcctatgggagaggcctcaccgtggctatggttacacaccgagaagccgagtccaagttagagtcaatgattgaacaacaaaaggaagaaaattttaaggaacaaggctaacccggtagtggagtgcctcatccagctaggatgcgtgcctacctgagggttggactcggctgatgggtgggtccgagcctatgcgaaggggccggatggagtactaggtcccctttgggagcttctcgtgctgtcgctcggtgagctcctcggtcggcgtcttagcggcacgcttgtcatcaacctggggaagtgcgggagtgttgggggggagagagacatacttctcatgaacctgtcacagcaaatttggagaaatcaagaaatcaagactcgaatgaagcaaatacagagattaaccactcagatcttatctttgaacagggtcagcggaggttgtacatgctagggccgttcttgtactcagccaccagctctgcagggatctgactggcctcaaagaattcgtcgaccagtctctccaactctgcttctaggaggttctctgtggactccctcgttgggctcttcaactcccgcaaagtcaaactctggatgcactcggctcttgatgggagcgatgcgtggatcaggcaactcactaaaatgctcgtaggcgtgagtcctgccttcttctccttcaggatcttgatgagatttggcacttgggccgacccgacagcagaaggaagaagggtccagtaaccgtgaggagttggagactggctagaaaagggggagggaaccgtcaatgttcttgacgtaaaacgactcggtgttctagcttttgttcgagctcttccaaataagtgcttttttaacctctcacagagctagaagccagctccccccacaagattggggctcacccaagtggggtggggtttaagggtgaaaagaaattgaaagaaagccaagtgtggcagaatgcccaagaacgcttcgcaaatgtggataaaaagcacaaatgtgcgaaatggagtttgggttgaggtgatgagcctcgagcccatagtaatcaagaaggccacgaaagaagggagagattgggatcttgaatccacgtgggcaaaaggactcgaggtgtagtacctcgtttgtgtctggcgtggggacaacctctgcttccgcgggccgccagttaacaacctccttcaccgacaaatggtcacgctcgaccaatgtcctgagtctcccatcggagctcacagacatcagccatgtcctgtcctcctcagatgttgccttcgaagccttcaccaccttcttgctgcgacccatggtccgattggtggaggtggttgaatggggcggagagggtcttggttcaagagagagaaagagagaagatcacaagagctcaagaggcgtgggcgcaaggaggaagagaagagcaaatggaaaatatgctggcgtaatggacggaagggggccatttggggggatataaaggcgtgactacacgccccctcagctcccccgtcgccgttaatgacggagtatgccaaccgatcgttcacattagggttttagccttagtgggctttgatggaacgccacctcgacataatgaggcgtgtgaatcggggcgtggctacagtatttactaacatttatcagatcctagttgtcccaatatccgagaaaaagatttttgtcttactcgagaaggaacccaagactgccagctttttataagcattcgagtgaccacgactcagcccggtggcacaccttttgaggttgaggtacatgccactaccactagtaggatgatggtgccactcgggaacctctcaattgggaagctggtatgctctcgggagccgaatcactcgcttgataggtgtgactcggataccttacgaacaactcggctagagggtacgtacgatgtgctgatgactcggttctttaataaaagacgaggtgccctctaaagccttttatgggtcggattgtgagtccttcccaatcactctgccgacattaaaattgaccatgcgagattttttagcctacgagagcactcagctggtaagatgggttagcaacatcgggattgcgaaggactcaggggctgctgatgatgacacataacccgggtatctactagatgggcttaggccaccactcggatagggggtgggccgggccgccaggtcaagaccagctgctatacgactcggaataacactgccatgtgccccaaggtgtcggaagtccaagattagctaggctttctataaacctgttgtaatacgatacgaaagataggtgccgacttggctcaggactccgattgtaatcctgcctcttagactatataactaggggcagggtacccccttcaaacattcaatctcgtctccagcataagaaaacaatagccaccaaagcaggacatagggtattacgccattggcggcctgaacctgtctaaatcgtgtctctatgattttcctcgacaaccctacatataaatctactcgtctgcacaaagctatgacaactatatattgtaaaattactttcatgaagccccaagtaacataaactttgtattgtcaatatatgtaactatttatatatcgatgatattcacatgtaactaattttatggtcaatgttaaaaaaaaattgaatcagtaaacaacttatatttcagattggatggagtatgaaacagtggttgaaaatatctcaagcactgattgaagcatgacgacaaagcttccgattgcaataatgagaacatataggtggcatctcggctttctacgtggactaatgacaaagagaagatgcacatcatgccgcatataattcaatggactaatgatggtttatcggatcaagcattaacaatagacatcagtggattgggaaggcagacccagcatcttctttcatgagatagaacacgcaatggtgacccaaagtaagtatattctctctcgatttcttcctttcattccaggtccaaccaaatcatgatgttgtaggatgatgtgttacaccatcagaacaagaagatctttcttttttttttctcgagcgaggttagagcacatacaaggtataatatcagctgcctgtaagagttgccacgtaggatttctagcgatgcaaagaggttataatgaggagagagattgttgtctcttgacgaaatgactatctataatataagaaaagagtcgaaatgtgtcttgtaggatatctactatgaggcaacaacagtagattgtgggttatacaagctgtcatttgaattgtctaaaaatgacatgtacagtgtatagacagcagccatttcaactgttgtatgtatccttacagaatgatagaagtaaaactctcatgcaacaaatgcttgacaagggctctagagtttacgcggtgtgcatgcctagactcaactcataagctaccctatttggtcttgccatgtaaattaaatacggtaattattatcttgggatacgtgtcagaatatgatgtaaattattacttgttcaaaagaatatcttcaatttggcattgtcttttttcgttcgaactaactctgactgttaatgactatcaatacatactgacaggatgtacctatctttcttgatcggaagagatggacagttaaccttgaatatatttggacgacacatacattgcattattctttttgacggttgggagaagccgtaccataccgaatagccatgaaatatgttgggttcctaggacggttttaaaattctaatgtcgttttggctgtccctcaataaacccagctactcggtgataatctgaaagcgttacattgacacatgaataagacagcacaccacattgcattattctttttgacggctgggagaagccataccataccgaatagccatgaaatatgtcagattcgtagcacggttttaaaattctaatatcgttttggctgtccctcaataaacccagctactcggtgacattctgaaagcattacattgacacatgaatgagacagcacaccacattgcattattctttttgacggctgggagaagccataccataccgaatagccatgaaatatgttagattcctagcacggttttaaaattctaatactccctccgttcatttatctccatcattttagccttcggcgtagtgacccaaggagcagagtaaaaccactcagtttgtatttaatcatcgcgtgctgagcgcatacgaggagtcacaggttaggaacgcagtaaagagacaataaatgcaaatgagtggtagccaatgagcagcaaatagaaaataaatgcgggcaagtcccaagctaatatgaaggagatttttgaataaaaagtttttgcttagatgaaggagataaatgaatggagggaatattgttttagctgttcctcagtaaacccaactactcggtgacaatccgaaagcgttatattgacacatgactgagacagcacaccgtcctctttcccctatcgcttggctccatctccatcacttgccaggggccaggggccaggggccaaataggagcgggcaaataatcgacaaaaattacctctttaatcgaatccccaccctgccaaattccaaggattccttcctccagatataagagtcatcgcccttgggtttcttggccgcagctccttcagttggctcttggtcggcttcttgctgctcttcctgagagaacctcgaattccgcaagaatttaactcgctgttgcagggatcattgagctcacgtgcagggggaaaac

SEQ ID NO: 4 - DNA, 5084 bp, synthetic construct
  source 1..5084: mol_type = other DNA
ttaattaagaaaaggatggcactgaagcataaaaattaagactttaaggttaagtaggagagagctctacttacccgtcgaagtggcactggaaggtgggggacaccactcagagcgctagccttgagctttgcgctggtagagcggctcagagtggtattgctgccaccggtgcgtgcagactcgtcccgaaggtcaccggagggataaccggttgcgtcacctccatggcctgcgggagaggcctcaccgtggctatggttacacaccgagaagccgagtccaagttagagtcaatgattgaacaacaaaaggaagaaaattttaaggaacaaggctaacccggtagtggagtgcctcatccagctaggatggtgtgcctacctgagggttggacttggctgatgggtgggtccgagcctatgcgaaggggccctagagcggatggagtactaggtcccctttgggagcttctcgcgctatcgctcggtgagctcctcggtcagcgtcttagtggcacacttgtcaccaacctggggaagtgtgggagtgttagggggagagagacatacttctcatgaacctgtcacaacaaatttggagaaatcaagaaatcaagactcgaatgaagcaaatacagagattaaccactcagatcttatctttgaacagggtcggcggaggttgtacatgccagggccgttcttgtactcagccactagctctgcagggatctgactggcctcaaagaattcgtcaaccagtctctccaactatgcttctaggaggttctctgtggactccctcgttggctcttcaactcccgcaaagtcaaactctggatgcactcggctcttgatgggagcgatgcgtggatcaggcaactcactaaaatgctcataggcgtgagtcctgccttcttctccttcaggatcttgataagatttggcacttgggccgactcgatagcagaaggaagaagggtccagtaaccgtgaggagttggagactggctagaaaagggggagggaaccgtcaatgttcttgacgtaaaacgactcagtgttctagcttttgttcgagctcttccaaataagtgcttttttaacctctcacggagctggaagccagctccccccacaagattggggctcactcgagtggggtggggcttaagggtgaaaagaaattgaaagaaagccaagtgtggcggaatgcccaagaatgcttcgcaaatgtggataaaagcacaaatgtgcgaaatggagtttgggttgaggtgatgagcctcgagcccatagtaatcaagaaggccacgaaagaagggagagattgggatctcgaatccacgtgggcaaaaggactcgaggtgtagtacctcgtttgtgtctggtgtggggacaacctctgcttccgcgggccgccagttaacaacctccttcaccggcaaatggtcacgctcgaccaatgtcctgagtctcccatcggagctcacagacattggccatgtcctgtcctcctcggatgtcgccttcgaagccttcaccaccttcttactgcgacccatggtccgattggtggaggtggttgaatggggcggagagggttttggttcaagagagagaaagagagaagatcacaagagctcaagaggcgtgggtgcaaggaggaagagaagagcaaatggctgatatgctgacgtaatggacagaagggggccatttggggggatataaaggcgtgactacacgccccctcagctcccccgtcgccgttaatgacggagtatgccaaccgatcgttcacattagggttttagccttagtgggctttgatggaacgccacctcgacataatgaggcgtgtgaatcggggcgtggctacagtatttactgacatttagcagaccctagttgtcccaatatccgagaaaaagatttttgtcttactcgagaaggaacccaagactgccagctttttataagcattcgggtgaccacgactcagcccggtggcacaccttttgaggttgaggtacatgccactaccactagtaggatgatggtgccactcgggaacctctcaattgggaagctggtatgctctcgggagccaaatcactcgcttgataggtgtgactcggataccttacgaacaactcggctagagggtacgtacgatgtgctgatgactcggttctttaataaaagacgaggtgccctctaaagccttttatgggtcggattgtgagtccttcccaatcactctgccgacattaaaattgaccatgcgagattttttagcctacgagagcacttggctggtaagatgggttagcaacatcgggattgcgaaggactcaggggctgctaatgatgacacataacccgggtatctactagatgggcttaggccaccactcggatagggggtgggccgggccgccaggtcaagaccagctgctatacgactcggaataacactgccatgtgccccaaggtgtcggaagtccaagattagctaggctttctataaacctgttgtaatacgatacgaaagataggtgccgacttggctcaggactccgattgtaatcctgcctcttagactatataactaggggcagggtacccccttcaaacattcaatctcgtctccagcataagaaaacaatagccaccaaagcaggacatagggtattacgccattggcggcctgaacctgtctaaatcgtgtctctatgattttcctcgacaaccctacatataaatctactcgtctgcacaaagctatgacaactatatattgtaaaattactttcatgaagccccaagtaacataaactttgtattgtcaatgtatgtaactatttatatatcgatgatattcacatgtaactaattttatggtcaatgttaaaaaaaattgaatcagtaaacaacttatatttcagattggatggagtatgaaacagtggttgaaaatatctcaagcactgattgaagcatgtacgacaaagcttccgattgcaataatgagaacatataggtggcatctcggctttctacgtggactaatgacaaagagaagatgcacatcatgccgcatataattcaatggactaatgatggtttatcggctcaagcattaacaatagacatcagtggattgggaaggcagacccagcatcttctttcatgagatagaacacgcaatggtgacccaaagtaagtatattctctctcgatttcttcctttcattccaggtccaaccaaatcatgatgttgtaggatgatgtgttacaccatcagaacaagaagatctttctttttttttttctcgagcgaggttagagcacatacaaggtataatatcagctgcctgtaagagttgccacgtaggatttctagcgatgcaaagaggttataatgaggagagagattgttgtctcttgacgaaatgactatctataatataagaaaagagtcgagatgtgtcttgtaggatatctactatgaggcaacaacaatagattgtgggttatacaagctgtcatttgaattgtctaaaaatgacatgtacagtgtatagacagcagccattttcaactgttgtatgtatccttacagaatgatagaagtaaaactctcatgcaacaaatgcttgacaagggctctagagtttacgcggtgtgcatgcctagactcaactcataagctaccctatttggtcttgccatgtaaattaaatacggtaattattatcttgggatacatgtcagaatatgatgtaattattacttgttcaaaagaatatcttcaatttggcattgtctttttcgttcgaactaactctgactgttaatgaactatcaatacatactgacaggatgtacctatctttcttgatcggaagagatggacagttaaccttgaatatatttggacgacacatacattgcattattctttttgacggttgggagaagccgtaccataccgaatagccatgaaatatgttaggttcctagcacggttttaaaattctaatgtcgttttggctgtccctcaataaacccagctactcggtgataatctgaaagcgttacattgacacatgaataagacagcacaccacattgcattattctttttgacggctgggagaagccataccataccgaatagccatgaaatatgtcagattcgtagcacggttttaaaattctaatatcgttttggctgtccctcaataaacccagctactcggtgacattctgaaagcattacattgacacatgaatgagacagcacaccacattgcattattctttttgacggctgggagaagccataccataccgaatagccatgaaatatgttagattcctagcacggttttaaaattctaatactccctccgttcatttatctccatcattttagccttcggcgtagtgaccaaggagcagagtaaaaccactcagtttgcatttaatcattgcgtgctgagcgcatacgaggagtcacaggttaggaacaaccaatgaacagtaaagagacaataaatgcaaacgggtggtagccaatgagcagcaaacagaaaataaatgcgggcaagtcccaagctaatatgaaggagatttttgaataaaaagtttttgcttagatgaaggagataaataaatggagggaatatcgttttagctgtccctcaataaacccaactactcggtgacaatccgaaagcgttatattgacacatgactgagacagcacaccatcctctttcccctatcgcttggctccatctccatcacttcccaggggccaggggccaaataggagcgggcaaataatcgacaaaaattacttctttaatcgaatccccaccctgccaaattccaaggattccttcctccagatataagagtcatcgtccttgggtttcttggccgcagctccttcagttggctcttggtcggcttcttgcccattactgctcttcctgagagaacctcgaattccgcaagaatttaactcgctgttgcagggatcgttgagctcacgtgcagggggaaaac

SEQ ID NO: 5 - DNA, 5084 bp, Saccharum sp.
  source 1..5084: mol_type = genomic DNA
ttaattaagaaaaggatggcactgaagcataaaaattaagactttaaggttaagtaggagagagctctacttacccgtcgaagtggcactggaaggtgggggacaccactcagagcgctagccttgagctttgcgctggtagagcggctcagagtggtattgctgccaccggtgcgtgcagactcgtcccgaaggtcaccggagggataaccggttgcgtcacctccatggcctgcgggagaggcctcaccgtggctatggttacacaccgagaagccgagtccaagttagagtcaatgattgaacaacaaaaggaagaaaattttaaggaacaaggctaacccggtagtggagtgcctcatccagctaggatggtgtgcctacctgagggttggacttggctgatgggtgggtccgagcctatgcgaaggggccctagagcggatggagtactaggtcccctttgggagcttctcgcgctatcgctcggtgagctcctcggtcagcgtcttagtggcacacttgtcaccaacctggggaagtgtgggagtgttagggggagagagacatacttctcatgaacctgtcacaacaaatttggagaaatcaagaaatcaagactcgaatgaagcaaatacagagattaaccactcagatcttatctttgaacagggtcggcggaggttgtacatgccagggccgttcttgtactcagccactagctctgcagggatctgactggcctcaaagaattcgtcaaccagtctctccaactatgcttctaggaggttctctgtggactccctcgttggctcttcaactcccgcaaagtcaaactctggatgcactcggctcttgatgggagcgatgcgtggatcaggcaactcactaaaatgctcataggcgtgagtcctgccttcttctccttcaggatcttgataagatttggcacttgggccgactcgatagcagaaggaagaagggtccagtaaccgtgaggagttggagactggctagaaaagggggagggaaccgtcaatgttcttgacgtaaaacgactcagtgttctagcttttgttcgagctcttccaaataagtgcttttttaacctctcacggagctggaagccagctccccccacaagattggggctcactcgagtggggtggggcttaagggtgaaaagaaattgaaagaaagccaagtgtggcggaatgcccaagaatgcttcgcaaatgtggataaaagcacaaatgtgcgaaatggagtttgggttgaggtgatgagcctcgagcccatagtaatcaagaaggccacgaaagaagggagagattgggatctcgaatccacgtgggcaaaaggactcgaggtgtagtacctcgtttgtgtctggtgtggggacaacctctgcttccgcgggccgccagttaacaacctccttcaccggcaaatggtcacgctcgaccaatgtcctgagtctcccatcggagctcacagacattggccatgtcctgtcctcctcggatgtcgccttcgaagccttcaccaccttcttactgcgacccatggtccgattggtggaggtggttgaatggggcggagagggttttggttcaagagagagaaagagagaagatcacaagagctcaagaggcgtgggtgcaaggaggaagagaagagcaaatggctgatatgctgacgtaatggacagaagggggccatttggggggatataaaggcgtgactacacgccccctcagctcccccgtcgccgttaatgacggagtatgccaaccgatcgttcacattagggttttagccttagtgggctttgatggaacgccacctcgacataatgaggcgtgtgaatcggggcgtggctacagtatttactgacatttagcagaccctagttgtcccaatatccgagaaaaagatttttgtcttactcgagaaggaacccaagactgccagctttttataagcattcgggtgaccacgactcagcccggtggcacaccttttgaggttgaggtacatgccactaccactagtaggatgatggtgccactcgggaacctctcaattgggaagctggtatgctctcgggagccaaatcactcgcttgataggtgtgactcggataccttacgaacaactcggctagagggtacgtacgatgtgctgatgactcggttctttaataaaagacgaggtgccctctaaagccttttatgggtcggattgtgagtccttcccaatcactctgccgacattaaaattgaccatgcgagattttttagcctacgagagcacttggctggtaagatgggttagcaacatcgggattgcgaaggactcaggggctgctaatgatgacacataacccgggtatctactagatgggcttaggccaccactcggatagggggtgggccgggccgccaggtcaagaccagctgctatacgactcggaataacactgccatgtgccccaaggtgtcggaagtccaagattagctaggctttctataaacctgttgtaatacgatacgaaagataggtgccgacttggctcaggactccgattgtaatcctgcctcttagactatataactaggggcagggtacccccttcaaacattcaatctcgtctccagcataagaaaacaatagccaccaaagcaggacatagggtattacgccattggcggcctgaacctgtctaaatcgtgtctctatgattttcctcgacaaccctacatataaatctactcgtctgcacaaagctatgacaactatatattgtaaaattactttcatgaagccccaagtaacataaactttgtattgtcaatgtatgtaactatttatatatcgatgatattcacatgtaactaattttatggtcaatgttaaaaaaaattgaatcagtaaacaacttatatttcagattggatggagtatgaaacagtggttgaaaatatctcaagcactgattgaagcatgtacgacaaagcttccgattgcaataatgagaacatataggtggcatctcggctttctacgtggactaatgacaaagagaagatgcacatcatgccgcatataattcaatggactaatgatggtttatcggctcaagcattaacaatagacatcagtggattgggaaggcagacccagcatcttctttcatgagatagaacacgcaatggtgacccaaagtaagtatattctctctcgatttcttcctttcattccaggtccaaccaaatcatgatgttgtaggatgatgtgttacaccatcagaacaagaagatctttctttttttttttctcgagcgaggttagagcacatacaaggtataatatcagctgcctgtaagagttgccacgtaggatttctagcgatgcaaagaggttataatgaggagagagattgttgtctcttgacgaaatgactatctataatataagaaaagagtcgagatgtgtcttgtaggatatctactatgaggcaacaacaatagattgtgggttatacaagctgtcatttgaattgtctaaaaatgacatgtacagtgtatagacagcagccattttcaactgttgtatgtatccttacagaatgatagaagtaaaactctcatgcaacaaatgcttgacaagggctctagagtttacgcggtgtgcatgcctagactcaactcataagctaccctatttggtcttgccatgtaaattaaatacggtaattattatcttgggatacatgtcagaatatgatgtaattattacttgttcaaaagaatatcttcaatttggcattgtctttttcgttcgaactaactctgactgttaatgaactatcaatacatactgacaggatgtacctatctttcttgatcggaagagatggacagttaaccttgaatatatttggacgacacatacattgcattattctttttgacggttgggagaagccgtaccataccgaatagccatgaaatatgttaggttcctagcacggttttaaaattctaatgtcgttttggctgtccctcaataaacccagctactcggtgataatctgaaagcgttacattgacacatgaataagacagcacaccacattgcattattctttttgacggctgggagaagccataccataccgaatagccatgaaatatgtcagattcgtagcacggttttaaaattctaatatcgttttggctgtccctcaataaacccagctactcggtgacattctgaaagcattacattgacacatgaatgagacagcacaccacattgcattattctttttgacggctgggagaagccataccataccgaatagccatgaaatatgttagattcctagcacggttttaaaattctaatactccctccgttcatttatctccatcattttagccttcggcgtagtgaccaaggagcagagtaaaaccactcagtttgcatttaatcattgcgtgctgagcgcatacgaggagtcacaggttaggaacaaccaatgaacagtaaagagacaataaatgcaaacgggtggtagccaatgagcagcaaacagaaaataaatgcgggcaagtcccaagctaatatgaaggagatttttgaataaaaagtttttgcttagatgaaggagataaataaatggagggaatatcgttttagctgtccctcaataaacccaactactcggtgacaatccgaaagcgttatattgacacatgactgagacagcacaccatcctctttcccctatcgcttggctccatctccatcacttcccaggggccaggggccaaataggagcgggcaaataatggacaaaaattacttctttaatcgaatgcccaccctgccaaattccaaggattccttcctccagatataagagtcatcgtccatgggtttcttggccgcagctccttcagttggctcttggtcggcttcttgcccattactgctcttcctgagagaacctcgaattccgcaagaatttaactcgctgttgcagggatcgttgagctcacgtgcagggggaaaac

SEQ ID NO: 6 - protein, 397 aa, Sorghum bicolor
MQSAAAFRPCPAGAGAGAGQLVSRNPSRGPLLPVPARPLRVVVSAATTRALGLGRLRLSASPDDRSGQRQVSCGAAGDAVAAPSAEEGGGFMKTLWLGSLFGLWYLFNIYFNIYNKQVLKVFPYPINITEAQFAVGSVVSLFFWTTGIIKRPKISGAQLAAILPLAIVHTMGNLFTNMSLGKVAVSFTHTIKAMEPFFSVLLSAIFLGEFPTVWVVASLLPIVGGVALASLTEASFNWIGFWSAMASNVTFQSRNVLSKKLMVKKEESLDNLNLFSIITVMSFFVLAPVTFFTEGVKITPTFLQSAGLNVNQVLTRSLLAGLCFHAYQQVSYMILAMVSPVTHSVGNCVKRVVVIVTSVLFFRTPVSPINSLGTAIALAGVFLYSQLKRLKPKPKTP

SEQ ID NO: 7 - protein, 329 aa, Sorghum bicolor
MTTPSFLVGIAGNVISILVFASPIATFRRIVRNKSTGDFTWLPYVTTLLSTSLWTFYGLLKPKGLLVVTVNGAGAALEAVYVTLYLVYAPRETKAKMGKLVLAVNVGFLAVVVAVALLALHGGARLDAVGLLCAAITIGMYAAPLGSMRTVVKTRSVEYMPFSLSFFLFLNGGVWSVYSLLVRDYFIGVPNAVGFVLGTAQLVLYLAFRNKAAERKDDDDEKEAAAAAPSSGDEEEGLAHLMGPPQVEMEMTAQQRGRLRLHKGQSLPKPPTGGPLSSSSSSSPHHGFGSIIKSLSATPVELHSVLYQHGLGRGRFEPVKKDDVDATNH

(19) United States
(12) Patent Application Publication
(10) Pub. No.: US 2025/0270574 A1
(43) Pub. Date: Aug. 28, 2025

(54) STEM CELL-SPECIFIC PROMOTER FROM SACCHARUM AND METHODS FOR INCREASING FATTY ACID AND/OR OIL ACCUMULATION

(71) Applicant: The Board of Trustees of the University of Illinois, Urbana, IL (US)

(72) Inventors: Li-Qing CHEN, Champaign, IL (US); Jiang WANG, Urbana, IL (US)

(21) Appl. No.: 19/065,181

(22) Filed: Feb. 27, 2025

Related U.S. Application Data
(60) Provisional application No. 63/558,768, filed on Feb. 28, 2024.

(51) Int. Cl. C12N 15/82; C07K 14/415

(52) U.S. Cl. CPC C12N 15/8247; C07K 14/415; C12N 15/8226

(57) ABSTRACT

Compositions and methods for increasing fatty acid and/or oil accumulation in the stems of plants are provided. Constructs including stem cell-specific promoters operably linked to nucleic acids encoding a plastidic phosphoenolpyruvate/phosphate translocator protein and a tonoplast-localized hexose transporter protein are described.

### CROSS-REFERENCE TO RELATED APPLICATIONS

This application claims benefit of U.S. Provisional Patent Application Ser. No. 63/558,768, filed Feb. 28, 2024, the content of which is incorporated herein by reference in its entirety.

### INTRODUCTION

This invention was made with government support under DE-SC0018254 and DE-SC0018420 awarded by the Department of Energy. The government has certain rights in the invention.

### REFERENCE TO AN ELECTRONIC SEQUENCE LISTING

The contents of the electronic sequence listing (name: IL0045US_ST26.xml; size: 32,787 bytes; and date of creation: Feb. 27, 2025) is herein incorporated by reference in its entirety.

### BACKGROUND

Bioenergy production from feedstock crops provides a promising alternative to fossil fuels while reducing carbon emissions and helping mitigate global warming. oilseeds, such as soybean and canola, are the main sources for bio-oil production, which compete for oil crop supply. Non-oil plants normally do not accumulate high levels of fatty acids or oils (in the form of triacylglycerol (TAG)), particularly in their vegetative tissues. However, considerable progress in improving TAG accumulation has been made in the metabolic engineering of oil and fatty acids biosynthesis in vegetative tissues of high biomass biofuel feedstocks. The current engineering strategy for lipid hyper-accumulation mainly involves the overexpression of three key lipogenic factors: WRINKLED1 (WRI1), a master regulator for fatty acids biosynthesis; DIACYLGLYCEROL ACYLTRANSFERASE1 (DGAT1), a key enzyme that catalyzes the last step of TAG formation from diacylglycerol (DAG); and OLEOSIN1 (OLE1), a structural protein that stabilizes lipid droplets and protects it from being degraded. Despite these successes, additional strategies are needed to further enhance lipid accumulation in vegetative tissues of bioenergy feedstocks.

The phosphoenolpyruvate (PEP) is a glycolytic intermediate that serves as a precursor for several important pathways, including fatty acid (FA) biosynthesis and shikimate pathway in chloroplast/plastids. The plastidic PEP, in principle, has three supply routes: (1) plastidic glycolysis from 3-phosphoglycerate (3-PGA) involving phosphoglyceromutase (PGyM) and enolase (ENO); (2) import from cytosol via PEP/phosphate translocator (PPT); (3) conversion from plastidic pyruvate by pyruvate orthophosphate dikinase (PPDK). Although plastidic pyruvate can be imported from cytosol via Bile Acid:Sodium Symporter family protein 2 (BASS2), it is not clear whether this route occurs at a rate sufficient for PEP synthesis through PPDK. In chloroplast or most non-green plastids (except from lipid-storing seeds), PEP import from cytosol via PPT has been proposed to be the main route of supply due to the lack of a complete plastidic glycolytic pathway. In Arabidopsis, two PPT genes, PPT1 and PPT2, were identified, and the mutant of PPT1, also known as chlorophyll a/b-binding protein underexpressed 1 (cue1), exhibits a reticulate leaf phenotype and defect in chloroplast development, which can be fully rescued by PPT1 but only partially rescued by PPT2 or PPDK, suggesting the import of PEP into chloroplast/plastid is important to plant growth and leaf development. However, whether the plastidic PEP influx can be engineered to boost FA biosynthesis has not been reported until recently. where constitutive overexpression of BnaPPT1 promoted seed oil accumulation in oilseed crop Brassica napus, while the FA content was not affected at maturity (Tang et al. (2022) J. Adv. Res. 42:29-40).

Sugarcane produces 40% of the world's biofuel and is the most productive crop per unit of arable land annually, with a total production of about 2.03 billion tons from an area of 27.4 million ha, resulting in an average fresh cane yield of 73.9 t/ha worldwide. Metabolic engineering of sugarcane to accumulate FA or oils has been described (Cao et al. (2023) GCB Bioenergy 15:1450-1464; Zale et al. (2016) Plant Biotechnol. J. 14:661-669; Maitra et al. (2024) Chem. Engineer. J. 487:150450; Kannan et al. (2022) Mol. Breed. 42:64; Luo et al. (2022) BMC Biotechnol. 22:24; Maitra et al. (2022) ACS Sustainable chem. Eng. 10:16833-16844; Parajuli et al. (2020) GCB Bioenergy 12:476-490). However, there is a need in the art to genetically manipulate this plant in a tissue- or cell-specific manner, e.g., in mature stem parenchyma cells where vast quantities of photoassimilates are stored and available for efficient oil conversion. However, the tools necessary for mature stem, parenchyma cell-specific expression are lacking.

Several promoters have been proposed to be stem-specific such as the pLSG and pA157 promoters (Moyle & Birch (2013) Plant Mol. Biol. 82(1-2):51-8; Mudge et al. (2013) Plant Biotechnology Journal 11(4):502-509). However, these promoters have failed to drive reporter gene specifically in the stem in sugarcane since they are also significantly expressed in roots or leaves. Further, it has not been shown that these stem preferred genes can be detected in the parenchyma cells rather than vascular cells. Two other promoters, pDIR16 and pOMT, can regulate the expression of a gene of interest specifically to the stem in sugarcane and rice, however, only in the vascular tissues rather than parenchyma cells (Damaj et al. (2010) Planta 231(6):1439-58). Stem parenchyma cell-specific genes encoding tonoplast sugar transporters have been described and promoters of the same have been shown to drive GUS reporter gene specifically in the Arabidopsis stem (Wang et al. (2021) GCB Bioenergy 13(9):1515-27.

### SUMMARY OF THE INVENTION

The present invention provides a construct for increasing fatty acid and/or oil synthesis in stem cells, wherein the construct is composed of (i) a first Saccharum Tonoplast Sugar Transporter (TST) promotor operably linked to nucleic acids encoding a plastidic phosphoenolpyruvate/phosphate translocator (PPT) protein; and (ii) a second Saccharum TST promotor operably linked to nucleic acids encoding a tonoplast-localized hexose transporter protein.

A transgenic plant or plant cell including the construct is also provided, as is a method for producing a genetically modified plant or plant cell having increased fatty acid and/or oil accumulation in stem cells comprising (a) transforming one or more host plants or plant cells with a construct of the invention; and (b) selecting one or more transformed plants or plant cells for increased fatty acid accumulation and/or oil in stem cells as compared to a control plant or plant cell lacking the construct.

A method for producing a genetically modified plant having increased fatty acid and/or oil accumulation is also provided, the method comprising overexpressing a plastidic phosphoenolpyruvate/phosphate translocator (PPT) protein in a tissue of a plant thereby producing the genetically modified plant having increased fatty acid and/or oil accumulation.

### BRIEF DESCRIPTION OF THE DRAWINGS

FIG. 1 shows total soluble sugar quantified from Arabidopsis leaves (21 days post-germination (DPG) collected in four independent repeats (means±SE, n=4). Soluble sugars (including glucose (Glc), fructose (Frc), and sucrose (Suc)) were quantified using HPLC equipped with a refractive index detector (RID) and provided on a dry weight (DW) basis. Leaves in were collected at the end of the light stage. The statistically significant differences among samples were determined using one-way ANOVA followed by multiple comparison tests (Fisher's LSD method) and were represented by different letters (P<0.05).

FIG. 2 shows total fatty acid quantified from Arabidopsis leaves (21 DPG) collected in five independent repeats (means±SE, n=5). Leaves in were collected at the end of the light stage. The statistically significant differences among samples were determined using one-way ANOVA followed by multiple comparison tests (Fisher's LSD method) and were represented by different letters (P<0.05). FW, fresh weight.

FIG. 3 shows total fatty acid quantification from sugarcane internode 15 (IN15; counting from top) using GC-FID. All tested IN15 samples were collected in three independent repeats (means±SE, n=3). The statistically significant differences among samples were determined using one-way ANOVA followed by multiple comparison tests (Fisher's LSD method) and were represented by different letters (P<0.05). DW, dry weight.

FIG. 4 shows the sum of triacylglycerol (TAG) species calculated from lipidomic analysis in sugarcane. All tested IN15 samples were collected in three independent repeats (means±SE, n=3). The statistically significant differences among samples were determined using one-way ANOVA followed by multiple comparison tests (Fisher's LSD method) and were represented by different letters (P<0.05). DW, dry weight.

### DETAILED DESCRIPTION OF THE INVENTION

Several highly expressed candidate genes with stem cell-specific expression were selected from the published sugarcane RNA-seq dataset. Real-time PCR analysis was conducted to confirm candidate gene expression levels across various tissues at two developmental stages (immature and mature) in both sugarcane and energycane. Two candidate genes, namely TST1 (Tonoplast Sugar Transporter 1) and TST2b, were strongly and almost exclusively expressed in the stem of both sugarcane and energycane. In addition, abundant TST1and TST2b RNA transcripts were found in the pith parenchyma cells of the mature stem using optimized RNA in situ hybridization with gene-specific RNA probes. The promoter regions (5000 bp upstream from ATG) of TST1 (pTST1-1P) and TST2b (including pTST2b-1A, and pTST2b-1C) were subsequently cloned and the activities of both pTST2b-1A and pTST2b-1C to drive the β-Glucuronidase (GUS) reporter gene exclusively in the stem was confirmed using Arabidopsis thaliana. The specific expression of pTST2b-1A in sugarcane stem was also confirmed. The data showed that pTST2b-1A is able to drive high level expression of the GUS reporter gene in the stem parenchyma cells of sugarcane stem, including mature, immature, and mid-mature stem sections. The level of expression in the mature stem exceeds the expression found with earlier reported stem specific (e.g., pA157) or constitutive (e.g., Zea mays ubiquitin) promoters.

In addition to the identification of promoters for selectively expressing a gene of interest in sugarcane stem, it has now been demonstrated that overexpression of plastidic phosphoenolpyruvate/phosphate translocator (PPT) protein can redirect phosphoenolpyruvate flux into plastids thereby boosting fatty acid levels in sugar-rich tissue. In particular, it was observed that overexpression of PPT in a sugar-rich mutant of Arabidopsis (sweet11;12;13 mutant) increased fatty acid and oil accumulation in older leaves. Based upon the analysis in Arabidopsis, a sorghum PPT protein was overexpressed in sugarcane using a TST2b promoter and it was likewise observed that fatty acid levels increased by 21% to 55% in mature internodes in a sugar-rich environment, i.e., in a transgenic sugarcane plants that overexpressed tonoplast-localized hexose transporter (SWEET16).

Using TST2b promoters and/or PPT overexpression, sugarcane and/or energycane can be precisely engineered to allow abundant non-structural carbohydrates stored in stem parenchyma cells to be diverted for oil biosynthesis and accumulation without negatively affecting photosynthesis in leaves and plant growth at early developmental stages, thereby increasing the biofuel production per hectare of land. The promoters described herein may also be used in other biofuel crops, such as sorghum and miscanthus, in which stems are the major carbohydrate sink tissues. In addition, the promoters may be used to engineer and improve other stem-related traits, such as stem thickness or hyper-accumulation of sugars or value-added products in the stem.

Accordingly, this invention provides a genetic construct(s) and use thereof for increased fatty acid and/or oil production and accumulation in the stem of a plant, in particular a sugarcane plant. Specifically, the invention provides a construct(s) composed of at least one TST2b promoter from Saccharum species in combination with nucleic acids encoding a PPT protein and optionally a tonoplast-localized hexose transporter (SWEET16, Sugar Will Eventually be Exported Transporter 16) protein for redirecting sugar flux toward fatty acid production in stem cells of plants. In some aspects, the invention provides a construct(s) composed of a first Saccharum TST promotor operably linked to nucleic acids encoding a PPT protein; and a second Saccharum TST promotor operably linked to nucleic acids encoding a SWEET16 protein.

In this application, the use of the singular includes the plural unless specifically stated otherwise. It must be noted that, as used in the specification, the singular forms “a,” “an,” and “the” include plural referents unless the context clearly dictates otherwise.

The term “construct,” “recombinant DNA construct,” “recombinant construct,” “expression construct,” or “expression cassette” refers to any agent such as a plasmid, cosmid, virus, (bacterial BAC artificial chromosome), autonomously replicating sequence, phage, or linear or circular single-stranded or double-stranded DNA or RNA nucleotide sequence, derived from any source, capable of genomic integration or autonomous replication, comprising a DNA molecule in which one or more DNA sequences have been operably linked in a functional manner using well-known recombinant DNA techniques.

A “promoter” refers to a nucleic acid sequence located upstream or 5′ to a translational start codon of an open reading frame (or protein-coding region) of a gene and that is involved in recognition and binding of RNA polymerase II and other proteins (trans-acting transcription factors) to initiate transcription. A promoter is said to “mediate” expression of a protein-coding sequence when expression is (e.g., transcription) of the protein-coding sequence dependent upon presence and/or sequence of the promoter.

A “stem cell-specific promoter” is a promoter that regulates expression of a coding sequence in a stem cell-specific manner. “Expression” of a coding sequence refers to the transcription of a coding sequence to produce the corresponding mRNA and translation of this mRNA to produce the corresponding gene product, i.e., a peptide, polypeptide, or protein. Expression of a coding sequence in a stem cell-specific manner means that the coding sequence is expressed only in the stem cells of a plant or that expression of the coding sequence is substantially restricted to the stem of a plant with negligible expression in other parts of the plant.

As used herein, the “stem” of a plant refers to the plant axis that bears buds and shoots and, at its basal end, roots. The stem conducts water, minerals, and food to other parts of the plant; it may also store food, and green stems themselves produce food. Plant stems, whether above or below ground, are characterized by the presence of nodes and internodes. Nodes are points of attachment for leaves, aerial roots, and flowers. The stem region between two nodes is called an internode.

A “stem cell” refers to a cell found in the stem of a plant, e.g., a parenchyma, collenchyma, and/or sclerenchyma cell. In some aspects, a plant stem cell is a parenchyma cell. In some aspects, the parenchyma cell is in the vascular tissue of a plant stem. In some aspects, the parenchyma cell is in the phloem and/or xylem tissue of a plant stem.

In accordance with this invention, constructs are provided that include Saccharum TST promotors. The TST promoters may be isolated from any Saccharum species including, but not limited to, S. spontaneum, S. officinarum L., S. barberi, S. sinense, S. edule, S. robustum, S. arundinaceum or interspecific hybrids thereof, e. g., energycane or sugarcane. An “isolated” nucleic acid sequence is substantially separated or purified away from other nucleic acid sequences with which the nucleic acid is normally associated in the cell of the organism in which the nucleic acid naturally occurs, i.e., other chromosomal or extrachromosomal DNA. The term embraces nucleic acids that are biochemically purified so as to substantially remove contaminating nucleic acids and other cellular components. The term also embraces recombinant nucleic acids and chemically synthesized nucleic acids.

A “Saccharum TST promotor” refers to a promoter that regulates expression of a Tonoplast Sugar Transporter in Saccharum. In some aspects, the TST promoter is from a TST1, TST2a, TST2b, TST3a, or TST3b gene. In some aspects, the Saccharum TST promotor is from a Saccharum TST2b gene. In some aspects, the Saccharum TST promotor is from a S. spontaneum TST2b gene. In some aspects, the Saccharum TST promotor is from an energycane TST2b gene. In some aspects, the Saccharum TST promotor is from a sugarcane TST2b gene. In some aspects, the TST promoter has a nucleotide sequence selected from any one of SEQ ID NOs: 1-5 (Table 1), or a fragment thereof that retains stem cell-specific promoter activity.

TABLE 1
>pTST2b-1A (S. spontaneum)
ttaattaaGCTAGCCTTGAGCTTTGCGCTGGCAGAGCGGCTCAGAGTGGTATTGCTGCCACCG
GTGCGTGCAGACTCGTCCCGAAGGTCACCGGAGGGATAACCGGTTGCGTCACCTCCATGGCCT
GTGGGAGAGGCCTCACCGTGGCTATGGTTACACACCGAGAAGCCGAGTCCAAGTTAGAGTCAA
TGATTGAACAACAAAAGGAAGAAAATTTTAAGGAACAAGGCTAACCCGGTAGTGGAGTGCCTC
ATCCAGCTAGGATGGCGTGCCTACCTGAGGGTTGGACTCGGCTGATGGGTGGGTCCGAGCCTA
TGCGAAGGGGCCCCAGAGCGGATGGAGTACTAGGTTCCCTTTGGGAGCTTCTCGCGATGTCGC
TCGGTGAGCTCCTCGGTCGGCATCTTAGCGGCACGCTTGTCATCAACCTGGGGAAGTGCGGGA
GTGTTGGGGGGAGAGAGACATACTTCTCATGAACCTGTCATAGCAAATTTGGAGAAATCAAGA
AATCAAGACTTGAATGAAGCAAATACAGAGATTAACCACTCAGATCTTATCTTTGAACAGGGT
CGGCGGAGGTTGTACATGCCAGGGCCGTTCTTGTACTCAGCCACCAGCTCTGCAGGGATCTGA
CTGGCCTCAAAGAATTCATCGACCAGTCTCTCCAACTCTGCTTCCAGGAGGTTCTCTGTGGAC
TCCCTCGTTGGCTCTTCAACTCCCGCAAAGTCAAACTCTGGATGCACTCGGCTCTTGATGGGA
GCGATGCGTGGATCAGGCAACTCACTAAAATGCTCGTAGGCGTGAGTCCTGCCTTCTTCTCCT
TCAGGATCTTGATGAGATTTGGCACTTGGGCCGACTCGACAGCAGAAGGAAGAAGGGTCCAAT
AACCATGAGGAGTTGGAGACTGGCTAGAAAAGGGGGAGGGAACCGTCAATGTTCTTGACGTAA
AACgACTCGGTGTTCTAGCTTTTGTTCGAGCTCTTCCAAATAAGTGCTTTTTTAACCTCTCAC
GGAGCTGGAAGCCAGCTCCCCCCACAAGATTGGGGCTCACCCAAGTGGGGTGGGGCTTAAGGG
TGAAAAGAAATTGAAAGAAAGCCAAGTGTGGCAGAATGCCCAAGAATGCTTTGCAAATGTGGA
TAAAAGCACAAATGTGCGAAATGGAGTTTGGGTTGAGGTGATGAGCCTCGAGCCCATAGTAAT
CAAGAAGGCCACGAAAGAAGGGAGAGATTGGGATCTCGAATCCACGTGGGCAAAAGGACTCGA
GGTGTAGTACCTCGTTTGTGTCTGGCGTGGGGACAACCTCTGCTTCCGCGGGCCGCCAGTTAA
CAACCTCCTTCACCGGCAAATGGTCACGCTCGACCAATGTCCTGAGTCTCCCATCGGAGCTCA
CAGACATCGGCCATGTCCTGTCCTCCTTGGATGTCGCCTTCGAAGCCTTCACCACCTTCTTGC
TGCGACCCATGGTCCGATTGGTGGAGGTGGTTGAATGGGGCGGAGAGGGTTTTGGTTCAAGAG
AGAGAAAGAGAGATCACAAGAGCTCAAGAGGCGTGGGCGCAAGGAGGAAGAgAAGAGCAAATG
GCTGATATGCTGGCGTAATGGACAGAAGGGGGCCATTTGGGGGGATATAAAGGTGTGACTACA
CGCCCCCTCAGCTACCCCGTCGCCGTTAATGACGGAGTATGCCAACCGATCGTTCACATTAGG
GTTTTAGCCTTAGTGGGCTTTGATGGAACGCCACCTCGACATAATGAGGCGTGTGAATCGGGG
CGTGGCTACAATATTTACTAACATTTATCAGATCCTAGTTGTCCCAATATCCGAGAAAAAGAT
TTTTGTCTTACTCGAGAAGGAACCCAAGACTGCTAGCTTTTTATAAGCATTCGAGTGACCACG
ACTCAGCCCGGTGGCACACCTTTTGAGGTTGAGGTACATGCCACTACCACTAGTAGGATGATG
GTGCCACTCGGGAACCTCTCAATTGGGAAGTTGGTATGCTCTTGGGAGCCGAATCACTCGCTT
GATAGGTGTGACTCGGATACCTTACGAACAACTCGGCTAGAGGGTACGTACGATGTGCTGATG
ACTCGGTTCTTTAATAAAAGACGAGGTGCCCTCTAAAGCCTTTTATGGGTCGGATTGTGAGTC
CTTCCCAATCACTCTGCCGACATTAAAATTGACCATGCGAGATTTTTTAGCCTACGAGAGCAC
TCAGCTGGTAAGATGGGTTAGCAACATCGGGATTGCGAAGGACTCAGGGGCTGCTGATGATGA
CACATAACCCGGGTATCTACTAGTGGGCTTAGGCCACCACTCGGATAGGGGTGGGCCGGGCCG
CCAGGTCAAGACCAGCTGCTATACGACTCGGAATAACACTGCCATGTGCCCCAAGGTGTCGGA
AGTCCAAGATTAGCTAGGCTTTTATAAACCTGTTGTAATACGATACGAAAGATAGGTGCCGAC
TTGGCTCAGGACTCCGATTGTAATCCTGCCTCTTAGACTATATAACTAGGGGCAGGGTACCCC
CTTCAAACATTCAATCTGTCTCCAGCATAAGAAAACAATAGCCACCAAAGCAGGACATAGGGT
ATTACGCCATTGGCGGCCTGAACCTGTCTAAATCGTGTCTCTATGATTTTCCTCGACAACCCT
ACATATAAATCTACTCGTCTGCACAAAGCTATGACAACTATATATTGTAAAATTACTTTCATG
AAGCCCCAAGTAACATAAACTTTGTATTGTCAATATATGTAACTATTTATATATCGATGATAT
TCACATGTAACTAATTTTATGGTCAATGTTAAAAAAAATTGAATCAGTAAACAACTTATATTT
CAGATTGGATGGAGTATGAAACAGTGGTTGAAAATATCTCAAGCACTGATTGAAGCATGACGA
CAAAGCTTCCGATTGCAATAATGAGAACATATAGGTGGCATCTCGGCTTTCTACGTGGACTAA
TGACAAAGAGAAGATGCACATCATGCCGCATATAATTCAATGGACTAATGATGGTTTATCGGC
TCAAGCATTAACAATAGACATCAGTGGATTGGGAAGGCAGACCCAGCATCTTCTTTCATGAGA
TAGAACACGCAATGTGACCCAAAGTAAGTATATTCTCTCTCGATTTCTTCCTTTCATTCCAGG
TCCAACCAAATCATGATGTTGTAGGATGATGTGTTACACCATCAGAACAAGAAGATCTTTCTT
TTTTTTTCTCGAGCGAGGTTAGAGCACATACAAGGTATAATATCAGCTGCCTGTAAGAGTTGC
CACGTAGGATTTCTAGCGATGCAAAGAGGTTATAATGAGGAGAGAGATTGTTGTCTCTTGACG
AAATGACTATCTATAATATAAGAAAGAGTCGAGATGTGTCTTGTAGGATATCTACTATGAGGC
AACAACAATAGATTGTGGGTTATACAAGCTGTCATTTGAATTGTCTAAAAATGACATGTACAG
TGTATAGACAGCAGCCATTTCAACTGTTGTATGTATCCTTACAGAATGATAGAAGTAAAACTC
TCATGCACCAAATGCTTGACAAGGGCTCTAGAGTTTACGCGGTATGCAGGCCTAGACTCAACT
CATAAGCTACCCTATTTGGTCTTGCCATGTAAATTAAATACGGTAATTATTATCTTGGGATAC
ATGTCAGAATATGATGTAATTATTACTTGTTCAAAAGAATATCTTCAATTTGGCATTGTCTTT
TTCGTTCGAACTAACTCTGACTGTTAATGACTATCAACACATACTGACAGGATGTACCTATCT
TTCTTGATCGGAAGAgATGGACAGTTAACCTTGAATATATTTGGACGACACATACATTGCATT
ATTCTTTTTGACGGCTGGGAGAAGCCGTACCCTACCGAATAGCCATGAAATATGTTAGGTTCC
TAGGACGGTTTTAAAATTCTAATGTCGTTTTGGCTGTCCCTCAATAAACCTAGCTACTCGGTG
ATAATCTGAAAGCGTTACATTGACACATGAATAAGACAGCACACCACATTGCATTATTCTTTT
TGACGGCTGGGAGAAGCCATACCATACCGAATAGCCATGAAATATGTCAGATTCGTAGCACGG
TTTTAAAATTCTAATATCGTTTTGGCTGTCCCTCAATAAACCCAGCTACTCGGTGACATTCTG
AAAGCATTACATTGACACATGAATGAGACAGCACACCACATTGCATTATTCTTTTTGACGGCT
GGGAGAAGTCATACCATACCGAATAGCCATGAAATATGTTAGATTCCTAGCACGGTTTTAAAA
TTCTAATACTCCCTCCGTTCATTTATCTCCATCATTTTACTCTTCGGCGTAGTGACCAAGGAG
CAGAGTAAAACCACTCAGTTTGCATTTAATCATCGCGTGCTGAGCGTATACGAGGAGTCACAG
GTTAGGAACGCAGTAAAGAGACAATAAATACAAACGGGTGGTAGCCAATGAGCAGCAAACAGA
AAATAAATGCGGGCAAGTCCCAAGCTAATATGAAGAAGATTTTTAAATAAAAAGTTTTTGCTT
AGATGAAGGAGATAAATAAATGGAGGGAATATCGTTTTAGCTGTCCCTCAATAAACCCAACTA
CTCGGTGACAATCCGAAAGCGTTATATTGACACATGACTGAGACAGCACACCATCCTCTTTCC
CCTATCGCTTGGCTCCATCTCCATCACTTCCCAGGGGCCAGGGGCCAAATAGGAGCGGGCAAA
TAATGGACAAAAATTACTTCTTTAATCGAATGCCCACCCTGCCAAATTCCAAGGATTCCTTCC
TCCAGATATAAGAGTCATCGTCCATGGGTTTCTTGGCCGCAGCTCCTTCAGTTGGCTCTTGGT
CGGCTTCTTGCCCATTACTGCTCTTCCTGAGAGAACCTCGAATTCCGCAAGAATTTAACTCGC
TGTTGCAGGGACCGTTGAGCTCACGTGCAGGGGGAAAACgcggccgc (SEQ ID NO: 1)
>pTST2b-1C (S. spontaneum)
ttaattaaAAGGATGGCACTGAAGCATAAAAATTAAGACTTCAAGATTAAGTAGGAGAGAGCT
CTACTTACCTGTCGAAGCGGCGCTGGAAGGTGGGGGACACCACTCGGAGTGCTAGCCTTGAGC
TTTGCGCTGGCAGAGCAGCTCAGAGTGGTATTGCTGCCACCGGTGCGTGCAGACTCGTCCCGA
AGGTCACCGGAGGGATAACGGTTGCGTCACCTCCATGGCCTGTGGGAGAGGCCTCACCGTGGC
TATGGTTACACACCGAGAAGCCGAGTCCAAGTTAGAGTCAATGATTGAACAACAAAAGGAAGA
AAATTTTGCCTAACCCGGTAGTGGAGGATCCAGCTGAGGGTTGGACCTGAGGGTCCGAGCCTG
ATGGAGTACTAGGTCCCTTTGGGAGCTTCTCGTGCTGTCGCTCGGTGAGCTCCTCGGTCGGCG
TCTTAGCGGCACGCTTGTCATCAACCTGGGGAAGTGCGGGAGTGTTGGGGGGAGAGAGACATA
CTTCTCATGAACCTGTCACAGCAAATTTGGAGAAATCAAGAAATCAAGACTCGAATGAAGCAA
ATACAGAGATTAACCACTCAGATCTTATCTTTGAACAGGGTCGGCGGAGGTTGTACATGCTAG
GGCCGTTCTTGTACTCAGCCACCAGCTCTGCAGGGATCTGACTGGCCTCAAAGAATTCGTCGA
CCAGTCTCTCCAACTCTGCTTCTAGGAGGTTCTCTGTGGACTCCCTCGTTGGCTCTTCAACTC
CCGCAAAGTCAAACTCTGGATGCACTCGGCTCTTGATGGGAGCGATGCGTGGATCAGGCAACT
CACTAAAATGCTCGTAGGCATGAGTCCTGCCTTCTTCTCCTTTAGGATCTTGATGAGATTTGG
CACTTGGGCCGACTCGACAGCAGAAGGAAGAAGGGTCCAGTAACCATGAGGAGTTGGAGACTG
GCTAGAAAAGGGGGAGGGAACCGTCAATGTTCTTGACGTAAAACgACTCGGTGTTCTAGCTTT
TGTTCGAGGTCTTqCAAATAAGTGCTTTTTTAACCTCTCACAGAGCTGGAAGCCAGCTCCCCC
ACAAGATTGGGGCTCACCCAAGTGGGGTGGGGCTTAAGGGTGAAAAGAAATTGAAAGAAAGCC
AAGTGTGGCAGAATGCCCAAGAACGCTTCGCAAATGTGGATAAAAAGCACAAATGTGCGAAAT
GGAGTTTGGGTTGAGGTGATGAGCCTCGAGCCCATAGTAATCAAGAAGGCCACGAAAGAAGGG
AGAGATTGGGATCTCGAATCCACGTGGGCAAAAGGACTCGAGGTGTAGTACCTCGTTTGTGTC
TGGCGTGGGGACAACCTCTGCTTCCGCGGGCCGCCAGTTAACAACCTCCTTCACCGGCAAATG
GTCACGCTCGACCAATGTCCTGAGTCTCCCATCGGAGCTCACAGACATCGGCCATGTCCTGTC
CTCCTCAGATGTCGCCTTTGAAGCCTTCACCACCTTCTTGCTGCGACCCATGGTCCGATTGGT
GGAGGTGGTTGAATGGGGTGGAGAGGGTTTTGGTTCAAGAGAGAGAAAGAGAGAAGATCACAA
GAGCTCAAGAGGCGTGGGCGCAAGGAGGAAGAgAAGAGCAAATGGCTGATATGCTGGCGTAAT
GGACAGAAGGGGGCCATTTGGGGGGATATAAAGGCGTGACTACACGCCCCCTCAGCTCCCCCG
TCGCCGTTAATGACGGAGTATGCCAACCGATCGTTCACATTAGGGTTTTAGCCTTAGTGGGCT
TTGATGGAACGCCACCTCGACATAATGAGGCGTGTGAATCGGGGCGTGGCTACAGTATTTACT
AACATTTATCAGATCCTAGTTGTCCCAATATCCGAGAAGCATTCGAGTGACCACGACTCAGCC
CGGTGGCACACCTTTTGAGGTTGAGGTACATGCCACTACCACTAGTAGGATGATGGTGCCACT
CGGGAACCTCTCAATTGGGAAGCTGGTATGCTCTCGGGAGCCGAATCACTCGCTTGATAGGTG
TGACTCGGATACCTTACGAACAACTCGGCTATAGGGTACGTACGATGTGCTGATGACTCGGTT
CTTTAATAAAAGACGAGGTGCCCTCTAAAGCCTTTTATGGGTCGGATTGTGAGTCCTTCCCAA
TCACTCTGCCGACATTAAAATTGACCATGCGAGATTTTTTAGCCTACGAGAGCACTCAGCTGG
TAAGATGGGTTAGCAACATCGGGATTGCGAAGGACTCAGGGGCTGCTGCTGATGACACATAAC
CCGGGTATCTACTAGATGGGCTTAGGCCACCACTCGGATAGGGGGTGGGCCAGGCCGCCAGGT
CAAGACCAGCTGCTATACGACTCGGAATAACACTGCCATGTGCCCCAAGGTGTCGGAAGTCCA
AGATTAGCTAGGCTTTCTATAAACCTGTTGTAATACGATACGAAAGATAGGTGCCGACTTGGC
TCAGGACTCCGATTGTAATCCTGCCTCTTAGACTATATAACTAGGGGCAGGGTACCCCCTTCA
AACATTCAATCTCGTCTCCAGCATAAGAAAACAATAGCCACCAAAGCAGGACATAGGGTATTA
CGCCATTGGCGGCCTGAACCTGTCTAAATCGTGTCTCTATGATTTTCCTCGACAACCCTACAT
ATAAATCTACTCGTCTGCACAAAGCTATGACAACTATATATTGTAAAATTACTTTCATGAAGC
CCCAAGTAACATAAACTTTGTATTGTCAATATATGTAACTATTTATATATCGATGATATTCAC
ATGTAACTAATTTTATGGTCAATGTTAAAAAAAATTGAATCAGTAAACAACTTATATTTCAGA
TTGGATGGAGTATGAAACAGTGGTTGAAAATATCTCAAGCACTGATTGAAGCATGACGACAAA
GCTTCCGATTGCAATAATGAGAACATATAGGTGGCATCTCGGCTTTCTACGTGGACTAATGAC
AAAGAGAAGATGCACATCATGCCGCATATAATTCAATGGACTAATGATGGTTTATCGGCTCAA
GCATTAACAATAGACATCAGTGGATTGGGAAGGCAGACCCAGCATCTTCTTTCATGAGATAGA
ACACGCAATGGTGACCCAAAGTAAGTATATTCTCTCTCGATTTCTTCCTTTCATTCCAGGTCC
AACCAAATCATGATGTTGTAGGATGATGTGTTACACCATCAGAACAAGAAGATCTTTCTTTTT
TTTTTTCTCGAGCGAGGTTAGAGCACATACAAGGTATAATATCAGCTGCCTGTAAGAGTTGCC
ACGTAGGATTTCTAGCGATGCAAAGAGGTTATAATGAGGAGAGAGATTGTTGTCTCTTGACGA
AATGACTATCTATAATATAAGAAACGAGTCGAGATGTGTCTTGTAGGATATCTACTATGAGGC
AACAACAATAGATTGTGGGTTATACAAGCTGTCATTTGAATTGTCTAAAAATGACATGTACAG
TGTATAGACAGCAGCCATTTCAACTGTTGTATGTATCCTTACAGAATGATAGAAGTAAAACTC
TCATGCAACAAATGCTTGACAAGGGCTCTAGAGTTTACGCGGTGTGCATGCCTAGACTCAACT
CATAAGCTACCCTATTTGGTCTTGCCATGTAAATTAAATACGGTAATTATTATCTTGGGATAC
ATGTCAGAATATGATGTAATTATTACTTGTTCAAAAGAATATCTTCAATTTGGCATTGTCTTT
TTCGTTCGAACTAACTCTGACTGTTAATGACTATCAATACATACTGACAGGATGTACCTATCT
TTCTTGATCGGAAGAgATGGACAGTTAACCTTGAATATATTTGGACGACACATACATTGCATT
ATTCTTTTTGACGGTTGGGAGAAGCCGTACCATACCGAATAGCCATGAAATATGTTAGGTTCC
TAGGACGGTTTTAAAATTCTAATGTCGTTTTGGATGTCCCTCAATAAACCCAGCTACTCGGTG
ATAATCTGAAAGCGTTACATTGACACATGAATAAGACAGCACACCACATTGCATTATTCTTTT
TGACGGCTGGGAGAAGCCATACCATACCGAATAGCCATGAAATATGTCAGATTCGTAGCACGG
TTTTAAAATTCTAATATCGTTTTGGCTGTCCCTCAATAAACCCAGCTACTCGGTGACATTCTG
AAAGCATTACATTGACACATGAATGAGACAGCACACCACATTGCATTATTCTTTTTGACGGCT
GGGAGAAGCCATACCCTACCGAATAGCCATAAAATATGTTAGATTCCTAGCACGGTTTTAAAA
TTCTAATACTCCCTCCGTTCATTTATCTCCATCATTTTAGCCTTCGGCGTAGTGACCAAGGAG
CAGAGTAAAACCACTTAGTTTGCATTTAATCATCGCGTGCTGAGCGCATACGAGGAGTCACAG
GTTAGGAACGCAGTAAAGAGACAATAAATGCAAATGAGTGGTAGCCAATGAGCAGCAAACAGA
AAATAAATGCGGGCAAGTCCCAAGCTAATATGAAGGAGATTTTTGAATAAAAAGTTTTTGCTT
AGATGAAGGAGATAAATGAATGGAGAGAATATCGTTTTAGCTGTCCCTCAATAAACCCAACTA
CTCGGTGACAATCCGAAAGCGTTATATTGACACATGACTGAGACAGCACACCATCCTCTTTCC
CCTATCGCTTGGCTCCATCTCCATCACTTCCCAGGGGCCAGGGGCCAAATAGGAGCGGGCAAA
TAATGGACAAAAATTACTTCTTTAATCGAATGCCCACCCTGCCAAATTCCAAGGATTCCTTCC
TCCAGATATAAGAGTCATCGTCCATGGGTTTCTTGGCCGCAGCTCCTTCAGTTGGCTCTTGGT
CGGCTTCTTGCCCATTACTGCTCTTCCTGAGAGAACCTCGAATTCCGCAAGAATTTAACTCGC
TGTTGCAGGGATCATTGAGCTCACGTGCAGGGGGAAAACgcggccgc (SEQ ID NO: 2)
>pTST2b-1A (uATG modified from energycane cultivar UFCP82)
ttaattaaCGGCTCAGAGTGGTATTGCTGCCACCGGTGCGTGCAGACTCGTCCCGAAGGTCAC
CGGAGGGATAACCGGTTGCGTCGCCTCCATGGCCTATGGGAGAGGCCTCACCGTGGCTATGGT
TACACACCGAGAAGCCGAGTCCAAGTTAGAGTCAATGATTGAACAACAAAAGGAAGAAAATTT
TAAGGAACAAGGCTAACCCGGTAGTGGAGTGCCTCATCCAGCTAGGATGCGTGCCTACCTGAG
GGTTGGACTCGGCTGATGGGTGGGTCCGAGCCTATGCGAAGGGGCCGGATGGAGTACTAGGTC
CCCTTTGGGAGCTTCTCGTGCTGTCGCTCGGTGAGCTCCTCGGTCGGCGTCTTAGCGGCACGC
TTGTCATCAACCTGGGGAAGTGCGGGAGTGTTGGGGGGGAGAGAGACATACTTCTCATGAACC
TGTCACAGCAAATTTGGAGAAATCAAGAAATCAAGACTCGAATGAAGCAAATACAGAGATTAA
CCACTCAGATCTTATCTTTGAACAGGGTCAGCGGAGGTTGTACATGCTAGGGCCGTTCTTGTA
CTCAGCCACCAGCTCTGCAGGGATCTGACTGGCCTCAAAGAATTCGTCGACCAGTCTCTCCAA
CTCTGCTTCTAGGAGGTTCTCTGTGGACTCCCTCGTTGGGcTCTTCAACTCCCGCAAAGTCAA
ACTCTGGATGCACTCGGCTCTTGATGGGAGCGATGCGTGGATCAGGCAACTCACTAAAATGCT
CGTAGGCGTGAGTCCTGCCTTCTTCTCCTTCAGGATCTTGATGAGATTTGGCACTTGGGCCGA
CCCGACAGCAGAAGGAAGAAGGGTCCAGTAACCGTGAGGAGTTGGAGACTGGCTAGAAAAGGG
GGAGGGAACCGTCAATGTTCTTGACGTAAAACgACTCGGTGTTCTAGCTTTTGTTCGAGcTCT
TCCAAATAAGTGCTTTTTTAACCTCTCACAGAGCTAGAAGCCAGCTCCCCCCACAAGATTGGG
GCTCACCCAAGTGGGGTGGGGTTTAAGGGTGAAAAGAAATTGAAAGAAAGCCAAGTGTGGCAG
AATGCCCAAGAACGCTTCGCAAATGTGGATAAAAAGCACAAATGTGCGAAATGGAGTTTGGGT
TGAGGTGATGAGCCTCGAGCCCATAGTAATCAAGAAGGCCACGAAAGAAGGGAGAGATTGGGA
TCTTGAATCCACGTGGGCAAAAGGACTCGAGGTGTAGTACCTCGTTTGTGTCTGGCGTGGGGA
CAACCTCTGCTTCCGCGGGCCGCCAGTTAACAACCTCCTTCACCGACAAATGGTCACGCTCGA
CCAATGTCCTGAGTCTCCCATCGGAGCTCACAGACATCAGCCATGTCCTGTCCTCCTCAGATG
TTGCCTTCGAAGCCTTCACCACCTTCTTGCTGCGACCCATGGTCCGATTGGTGGAGGTGGTTG
AATGGGGCGGAGAGGGTCTTGGTTCAAGAGAGAGAAAGAGAGAAGATCACAAGAGCTCAAGAG
GCGTGGGCGCAAGGAGGAAGAgAAGAGCAAATGGAAAATATGCTGGCGTAATGGACGGAAGGG
GGCCATTTGGGGGGATATAAAGGCGTGACTACACGCCCCCTCAGCTCCCCCGTCGCCGTTAAT
GACGGAGTATGCCAACCGATCGTTCACATTAGGGTTTTAGCCTTAGTGGGCTTTGATGGAACG
CCACCTCGACATAATGAGGCGTGTGAATCGGGGCGTGGCTACAGTATTTACTAACATTTATCA
GATCCTAGTTGTCCCAATATCCGAGAAAAAGATTTTTGTCTTACTCGAGAAGGAACCCAAGAC
TGCCAGCTTTTTATAAGCATTCGAGTGACCACGACTCAGCCCGGTGGCACACCTTTTGAGGTT
GAGGTACATGCCACTACCACTAGTAGGATGATGGTGCCACTCGGGAACCTCTCAATTGGGAAG
CTGGTATGCTCTCGGGAGCCGAATCACTCGCTTGATAGGTGTGACTCGGATACCTTACGAACA
ACTCGGCTAGAGGGTACGTACGATGTGCTGATGACTCGGTTCTTTAATAAAAGACGAGGTGCC
CTCTAAAGCCTTTTATGGGTCGGATTGTGAGTCCTTCCCAATCACTCTGCCGACATTAAAATT
GACCATGCGAGATTTTTTAGCCTACGAGAGCACTCAGCTGGTAAGATGGGTTAGCAACATCGG
GATTGCGAAGGACTCAGGGGCTGCTGATGATGACACATAACCCGGGTATCTACTAGATGGGCT
TAGGCCACCACTCGGATAGGGGGTGGGCCGGGCCGCCAGGTCAAGACCAGCTGCTATACGACT
CGGAATAACACTGCCATGTGCCCCAAGGTGTCGGAAGTCCAAGATTAGCTAGGCTTTCTATAA
ACCTGTTGTAATACGATACGAAAGATAGGTGCCGACTTGGCTCAGGACTCCGATTGTAATCCT
GCCTCTTAGACTATATAACTAGGGGCAGGGTACCCCCTTCAAACATTCAATCTCGTCTCCAGC
ATAAGAAAACAATAGCCACCAAAGCAGGACATAGGGTATTACGCCATTGGCGGCCTGAACCTG
TCTAAATCGTGTCTCTATGATTTTCCTCGACAACCCTACATATAAATCTACTCGTCTGCACAA
AGCTATGACAACTATATATTGTAAAATTACTTTCATGAAGCCCCAAGTAACATAAACTTTGTA
TTGTCAATATATGTAACTATTTATATATCGATGATATTCACATGTAACTAATTTTATGGTCAA
TGTTAAAAAAAAATTGAATCAGTAAACAACTTATATTTCAGATTGGATGGAGTATGAAACAGT
GGTTGAAAATATCTCAAGCACTGATTGAAGCATGACGACAAAGCTTCCGATTGCAATAATGAG
AACATATAGGTGGCATCTCGGCTTTCTACGTGGACTAATGACAAAGAGAAGATGCACATCATG
CCGCATATAATTCAATGGACTAATGATGGTTTATCGGATCAAGCATTAACAATAGACATCAGT
GGATTGGGAAGGCAGACCCAGCATCTTCTTTCATGAGATAGAACACGCAATGGTGACCCAAAG
TAAGTATATTCTCTCTCGATTTCTTCCTTTCATTCCAGGTCCAACCAAATCATGATGTTGTAG
GATGATGTGTTACACCATCAGAACAAGAAGATCTTTCTTTTTTTTTTCTCGAGCGAGGTTAGA
GCACATACAAGGTATAATATCAGCTGCCTGTAAGAGTTGCCACGTAGGATTTCTAGCGATGCA
AAGAGGTTATAATGAGGAGAGAGATTGTTGTCTCTTGACGAAATGACTATCTATAATATAAGA
AAAGAGTCGAAATGTGTCTTGTAGGATATCTACTATGAGGCAACAACAGTAGATTGTGGGTTA
TACAAGCTGTCATTTGAATTGTCTAAAAATGACATGTACAGTGTATAGACAGCAGCCATTTCA
ACTGTTGTATGTATCCTTACAGAATGATAGAAGTAAAACTCTCATGCAACAAATGCTTGACAA
GGGCTCTAGAGTTTACGCGGTGTGCATGCCTAGACTCAACTCATAAGCTACCCTATTTGGTCT
TGCCATGTAAATTAAATACGGTAATTATTATCTTGGGATACGTGTCAGAATATGATGTAAATT
ATTACTTGTTCAAAAGAATATCTTCAATTTGGCATTGTCTTTTTTCGTTCGAACTAACTCTGA
CTGTTAATGACTATCAATACATACTGACAGGATGTACCTATCTTTCTTGATCGGAAGAgATGG
ACAGTTAACCTTGAATATATTTGGACGACACATACATTGCATTATTCTTTTTGACGGTTGGGA
GAAGCCGTACCATACCGAATAGCCATGAAATATGTTGGGTTCCTAGGACGGTTTTAAAATTCT
AATGTCGTTTTGGCTGTCCCTCAATAAACCCAGCTACTCGGTGATAATCTGAAAGCGTTACAT
TGACACATGAATAAGACAGCACACCACATTGCATTATTCTTTTTGACGGCTGGGAGAAGCCAT
ACCATACCGAATAGCCATGAAATATGTCAGATTCGTAGCACGGTTTTAAAATTCTAATATCGT
TTTGGCTGTCCCTCAATAAACCCAGCTACTCGGTGACATTCTGAAAGCATTACATTGACACAT
GAATGAGACAGCACACCACATTGCATTATTCTTTTTGACGGCTGGGAGAAGCCATACCATACC
GAATAGCCATGAAATATGTTAGATTCCTAGCACGGTTTTAAAATTCTAATACTCCCTCCGTTC
ATTTATCTCCATCATTTTAGCCTTCGGCGTAGTGACCCAAGGAGCAGAGTAAAACCACTCAGT
TTGTATTTAATCATCGCGTGCTGAGCGCATACGAGGAGTCACAGGTTAGGAACGCAGTAAAGA
GACAATAAATGCAAATGAGTGGTAGCCAATGAGCAGCAAATAGAAAATAAATGCGGGCAAGTC
CCAAGCTAATATGAAGGAGATTTTTGAATAAAAAGTTTTTGCTTAGATGAAGGAGATAAATGA
ATGGAGGGAATATTGTTTTAGCTGTTCCTCAGTAAACCCAACTACTCGGTGACAATCCGAAAG
CGTTATATTGACACATGACTGAGACAGCACACCGTCCTCTTTCCCCTATCGCTTGGCTCCATC
TCCATCACTTGCCAGGGGCCAGGGGCCAGGGGCCAAATAGGAGCGGGCAAATAATcGACAAAA
ATTACCTCTTTAATCGAATCCCCACCCTGCCAAATTCCAAGGATTCCTTCCTCCAGATATAAG
AGTCATCGCCCtTGGGTTTCTTGGCCGCAGCTCCTTCAGTTGGCTCTTGGTCGGCTTCTTGCT
GCTCTTCCTGAGAGAACCTCGAATTCCGCAAGAATTTAACTCGCTGTTGCAGGGATCATTGAG
CTCACGTGCAGGGGGAAAAC (SEQ ID NO: 3)
>pTST2b-1A (uATG modified from sugarcane cultivar CP88)
ttaattaaGAAAAGGATGGCACTGAAGCATAAAAATTAAGACTTTAAGGTTAAGTAGGAGAGA
GCTCTACTTACCCGTCGAAGTGGCACTGGAAGGTGGGGGACACCACTCAGAGCGCTAGCCTTG
AGCTTTGCGCTGGTAGAGCGGCTCAGAGTGGTATTGCTGCCACCGGTGCGTGCAGACTCGTCC
CGAAGGTCACCGGAGGGATAACCGGTTGCGTCACCTCCATGGCCTGCGGGAGAGGCCTCACCG
TGGCTATGGTTACACACCGAGAAGCCGAGTCCAAGTTAGAGTCAATGATTGAACAACAAAAGG
AAGAAAATTTTAAGGAACAAGGCTAACCCGGTAGTGGAGTGCCTCATCCAGCTAGGATGGTGT
GCCTACCTGAGGGTTGGACTTGGCTGATGGGTGGGTCCGAGCCTATGCGAAGGGGCCCTAGAG
CGGATGGAGTACTAGGTCCCCTTTGGGAGCTTCTCGCGCTATCGCTCGGTGAGCTCCTCGGTC
AGCGTCTTAGTGGCACACTTGTCACCAACCTGGGGAAGTGTGGGAGTGTTAGGGGGAGAGAGA
CATACTTCTCATGAACCTGTCACAACAAATTTGGAGAAATCAAGAAATCAAGACTCGAATGAA
GCAAATACAGAGATTAACCACTCAGATCTTATCTTTGAACAGGGTCGGCGGAGGTTGTACATG
CCAGGGCCGTTCTTGTACTCAGCCACTAGCTCTGCAGGGATCTGACTGGCCTCAAAGAATTCG
TCAACCAGTCTCTCCAACTATGCTTCTAGGAGGTTCTCTGTGGACTCCCTCGTTGGCTCTTCA
ACTCCCGCAAAGTCAAACTCTGGATGCACTCGGCTCTTGATGGGAGCGATGCGTGGATCAGGC
AACTCACTAAAATGCTCATAGGCGTGAGTCCTGCCTTCTTCTCCTTCAGGATCTTGATAAGAT
TTGGCACTTGGGCCGACTCGATAGCAGAAGGAAGAAGGGTCCAGTAACCGTGAGGAGTTGGAG
ACTGGCTAGAAAAGGGGGAGGGAACCGTCAATGTTCTTGACGTAAAACgACTCAGTGTTCTAG
CTTTTGTTCGAGCTCTTCCAAATAAGTGCTTTTTTAACCTCTCACGGAGCTGGAAGCCAGCTC
CCCCCACAAGATTGGGGCTCACTCGAGTGGGGTGGGGCTTAAGGGTGAAAAGAAATTGAAAGA
AAGCCAAGTGTGGCGGAATGCCCAAGAATGCTTCGCAAATGTGGATAAAAGCACAAATGTGCG
AAATGGAGTTTGGGTTGAGGTGATGAGCCTCGAGCCCATAGTAATCAAGAAGGCCACGAAAGA
AGGGAGAGATTGGGATCTCGAATCCACGTGGGCAAAAGGACTCGAGGTGTAGTACCTCGTTTG
TGTCTGGTGTGGGGACAACCTCTGCTTCCGCGGGCCGCCAGTTAACAACCTCCTTCACCGGCA
AATGGTCACGCTCGACCAATGTCCTGAGTCTCCCATCGGAGCTCACAGACATTGGCCATGTCC
TGTCCTCCTCGGATGTCGCCTTCGAAGCCTTCACCACCTTCTTACTGCGACCCATGGTCCGAT
TGGTGGAGGTGGTTGAATGGGGCGGAGAGGGTTTTGGTTCAAGAGAGAGAAAGAGAGAAGATC
ACAAGAGCTCAAGAGGCGTGGGTGCAAGGAGGAAGAgAAGAGCAAATGGCTGATATGCTGACG
TAATGGACAGAAGGGGGCCATTTGGGGGGATATAAAGGCGTGACTACACGCCCCCTCAGCTCC
CCCGTCGCCGTTAATGACGGAGTATGCCAACCGATCGTTCACATTAGGGTTTTAGCCTTAGTG
GGCTTTGATGGAACGCCACCTCGACATAATGAGGCGTGTGAATCGGGGCGTGGCTACAGTATT
TACTGACATTTAGCAGACCCTAGTTGTCCCAATATCCGAGAAAAAGATTTTTGTCTTACTCGA
GAAGGAACCCAAGACTGCCAGCTTTTTATAAGCATTCGGGTGACCACGACTCAGCCCGGTGGC
ACACCTTTTGAGGTTGAGGTACATGCCACTACCACTAGTAGGATGATGGTGCCACTCGGGAAC
CTCTCAATTGGGAAGCTGGTATGCTCTCGGGAGCCAAATCACTCGCTTGATAGGTGTGACTCG
GATACCTTACGAACAACTCGGCTAGAGGGTACGTACGATGTGCTGATGACTCGGTTCTTTAAT
AAAAGACGAGGTGCCCTCTAAAGCCTTTTATGGGTCGGATTGTGAGTCCTTCCCAATCACTCT
GCCGACATTAAAATTGACCATGCGAGATTTTTTAGCCTACGAGAGCACTTGGCTGGTAAGATG
GGTTAGCAACATCGGGATTGCGAAGGACTCAGGGGCTGCTAATGATGACACATAACCCGGGTA
TCTACTAGATGGGCTTAGGCCACCACTCGGATAGGGGGTGGGCCGGGCCGCCAGGTCAAGACC
AGCTGCTATACGACTCGGAATAACACTGCCATGTGCCCCAAGGTGTCGGAAGTCCAAGATTAG
CTAGGCTTTCTATAAACCTGTTGTAATACGATACGAAAGATAGGTGCCGACTTGGCTCAGGAC
TCCGATTGTAATCCTGCCTCTTAGACTATATAACTAGGGGCAGGGTACCCCCTTCAAACATTC
AATCTCGTCTCCAGCATAAGAAAACAATAGCCACCAAAGCAGGACATAGGGTATTACGCCATT
GGCGGCCTGAACCTGTCTAAATCGTGTCTCTATGATTTTCCTCGACAACCCTACATATAAATC
TACTCGTCTGCACAAAGCTATGACAACTATATATTGTAAAATTACTTTCATGAAGCCCCAAGT
AACATAAACTTTGTATTGTCAATGTATGTAACTATTTATATATCGATGATATTCACATGTAAC
TAATTTTATGGTCAATGTTAAAAAAAATTGAATCAGTAAACAACTTATATTTCAGATTGGATG
GAGTATGAAACAGTGGTTGAAAATATCTCAAGCACTGATTGAAGCATGTACGACAAAGCTTCC
GATTGCAATAATGAGAACATATAGGTGGCATCTCGGCTTTCTACGTGGACTAATGACAAAGAG
AAGATGCACATCATGCCGCATATAATTCAATGGACTAATGATGGTTTATCGGCTCAAGCATTA
ACAATAGACATCAGTGGATTGGGAAGGCAGACCCAGCATCTTCTTTCATGAGATAGAACACGC
AATGGTGACCCAAAGTAAGTATATTCTCTCTCGATTTCTTCCTTTCATTCCAGGTCCAACCAA
ATCATGATGTTGTAGGATGATGTGTTACACCATCAGAACAAGAAGATCTTTCTTTTTTTTTTT
CTCGAGCGAGGTTAGAGCACATACAAGGTATAATATCAGCTGCCTGTAAGAGTTGCCACGTAG
GATTTCTAGCGATGCAAAGAGGTTATAATGAGGAGAGAGATTGTTGTCTCTTGACGAAATGAC
TATCTATAATATAAGAAAAGAGTCGAGATGTGTCTTGTAGGATATCTACTATGAGGCAACAAC
AATAGATTGTGGGTTATACAAGCTGTCATTTGAATTGTCTAAAAATGACATGTACAGTGTATA
GACAGCAGCCATTTTCAACTGTTGTATGTATCCTTACAGAATGATAGAAGTAAAACTCTCATG
CAACAAATGCTTGACAAGGGCTCTAGAGTTTACGCGGTGTGCATGCCTAGACTCAACTCATAA
GCTACCCTATTTGGTCTTGCCATGTAAATTAAATACGGTAATTATTATCTTGGGATACATGTC
AGAATATGATGTAATTATTACTTGTTCAAAAGAATATCTTCAATTTGGCATTGTCTTTTTCGT
TCGAACTAACTCTGACTGTTAATGAACTATCAATACATACTGACAGGATGTACCTATCTTTCT
TGATCGGAAGAgATGGACAGTTAACCTTGAATATATTTGGACGACACATACATTGCATTATTC
TTTTTGACGGTTGGGAGAAGCCGTACCATACCGAATAGCCATGAAATATGTTAGGTTCCTAGC
ACGGTTTTAAAATTCTAATGTCGTTTTGGCTGTCCCTCAATAAACCCAGCTACTCGGTGATAA
TCTGAAAGCGTTACATTGACACATGAATAAGACAGCACACCACATTGCATTATTCTTTTTGAC
GGCTGGGAGAAGCCATACCATACCGAATAGCCATGAAATATGTCAGATTCGTAGCACGGTTTT
AAAATTCTAATATCGTTTTGGCTGTCCCTCAATAAACCCAGCTACTCGGTGACATTCTGAAAG
CATTACATTGACACATGAATGAGACAGCACACCACATTGCATTATTCTTTTTGACGGCTGGGA
GAAGCCATACCATACCGAATAGCCATGAAATATGTTAGATTCCTAGCACGGTTTTAAAATTCT
AATACTCCCTCCGTTCATTTATCTCCATCATTTTAGCCTTCGGCGTAGTGACCAAGGAGCAGA
GTAAAACCACTCAGTTTGCATTTAATCATTGCGTGCTGAGCGCATACGAGGAGTCACAGGTTA
GGAACAACCAATGAACAGTAAAGAGACAATAAATGCAAACGGGTGGTAGCCAATGAGCAGCAA
ACAGAAAATAAATGCGGGCAAGTCCCAAGCTAATATGAAGGAGATTTTTGAATAAAAAGTTTT
TGCTTAGATGAAGGAGATAAATAAATGGAGGGAATATCGTTTTAGCTGTCCCTCAATAAACCC
AACTACTCGGTGACAATCCGAAAGCGTTATATTGACACATGACTGAGACAGCACACCATCCTC
TTTCCCCTATCGCTTGGCTCCATCTCCATCACTTCCCAGGGGCCAGGGGCCAAATAGGAGCGG
GCAAATAATcGACAAAAATTACTTCTTTAATCGAATCCCCACCCTGCCAAATTCCAAGGATTC
CTTCCTCCAGATATAAGAGTCATCGTCCtTGGGTTTCTTGGCCGCAGCTCCTTCAGTTGGCTC
TTGGTCGGCTTCTTGCCCATTACTGCTCTTCCTGAGAGAACCTCGAATTCCGCAAGAATTTAA
CTCGCTGTTGCAGGGATCGTTGAGCTCACGTGCAGGGGGAAAAC (SEQ ID NO: 4)
>pTST2b-1A (original sugarcane cultivar CP88)
ttaattaaGAAAAGGATGGCACTGAAGCATAAAAATTAAGACTTTAAGGTTAAGTAGGAGAGA
GCTCTACTTACCCGTCGAAGTGGCACTGGAAGGTGGGGGACACCACTCAGAGCGCTAGCCTTG
AGCTTTGCGCTGGTAGAGCGGCTCAGAGTGGTATTGCTGCCACCGGTGCGTGCAGACTCGTCC
CGAAGGTCACCGGAGGGATAACCGGTTGCGTCACCTCCATGGCCTGCGGGAGAGGCCTCACCG
TGGCTATGGTTACACACCGAGAAGCCGAGTCCAAGTTAGAGTCAATGATTGAACAACAAAAGG
AAGAAAATTTTAAGGAACAAGGCTAACCCGGTAGTGGAGTGCCTCATCCAGCTAGGATGGTGT
GCCTACCTGAGGGTTGGACTTGGCTGATGGGTGGGTCCGAGCCTATGCGAAGGGGCCCTAGAG
CGGATGGAGTACTAGGTCCCCTTTGGGAGCTTCTCGCGCTATCGCTCGGTGAGCTCCTCGGTC
AGCGTCTTAGTGGCACACTTGTCACCAACCTGGGGAAGTGTGGGAGTGTTAGGGGGAGAGAGA
CATACTTCTCATGAACCTGTCACAACAAATTTGGAGAAATCAAGAAATCAAGACTCGAATGAA
GCAAATACAGAGATTAACCACTCAGATCTTATCTTTGAACAGGGTCGGCGGAGGTTGTACATG
CCAGGGCCGTTCTTGTACTCAGCCACTAGCTCTGCAGGGATCTGACTGGCCTCAAAGAATTCG
TCAACCAGTCTCTCCAACTATGCTTCTAGGAGGTTCTCTGTGGACTCCCTCGTTGGCTCTTCA
ACTCCCGCAAAGTCAAACTCTGGATGCACTCGGCTCTTGATGGGAGCGATGCGTGGATCAGGC
AACTCACTAAAATGCTCATAGGCGTGAGTCCTGCCTTCTTCTCCTTCAGGATCTTGATAAGAT
TTGGCACTTGGGCCGACTCGATAGCAGAAGGAAGAAGGGTCCAGTAACCGTGAGGAGTTGGAG
ACTGGCTAGAAAAGGGGGAGGGAACCGTCAATGTTCTTGACGTAAAACgACTCAGTGTTCTAG
CTTTTGTTCGAGCTCTTCCAAATAAGTGCTTTTTTAACCTCTCACGGAGCTGGAAGCCAGCTC
CCCCCACAAGATTGGGGCTCACTCGAGTGGGGTGGGGCTTAAGGGTGAAAAGAAATTGAAAGA
AAGCCAAGTGTGGCGGAATGCCCAAGAATGCTTCGCAAATGTGGATAAAAGCACAAATGTGCG
AAATGGAGTTTGGGTTGAGGTGATGAGCCTCGAGCCCATAGTAATCAAGAAGGCCACGAAAGA
AGGGAGAGATTGGGATCTCGAATCCACGTGGGCAAAAGGACTCGAGGTGTAGTACCTCGTTTG
TGTCTGGTGTGGGGACAACCTCTGCTTCCGCGGGCCGCCAGTTAACAACCTCCTTCACCGGCA
AATGGTCACGCTCGACCAATGTCCTGAGTCTCCCATCGGAGCTCACAGACATTGGCCATGTCC
TGTCCTCCTCGGATGTCGCCTTCGAAGCCTTCACCACCTTCTTACTGCGACCCATGGTCCGAT
TGGTGGAGGTGGTTGAATGGGGCGGAGAGGGTTTTGGTTCAAGAGAGAGAAAGAGAGAAGATC
ACAAGAGCTCAAGAGGCGTGGGTGCAAGGAGGAAGAgAAGAGCAAATGGCTGATATGCTGACG
TAATGGACAGAAGGGGGCCATTTGGGGGGATATAAAGGCGTGACTACACGCCCCCTCAGCTCC
CCCGTCGCCGTTAATGACGGAGTATGCCAACCGATCGTTCACATTAGGGTTTTAGCCTTAGTG
GGCTTTGATGGAACGCCACCTCGACATAATGAGGCGTGTGAATCGGGGCGTGGCTACAGTATT
TACTGACATTTAGCAGACCCTAGTTGTCCCAATATCCGAGAAAAAGATTTTTGTCTTACTCGA
GAAGGAACCCAAGACTGCCAGCTTTTTATAAGCATTCGGGTGACCACGACTCAGCCCGGTGGC
ACACCTTTTGAGGTTGAGGTACATGCCACTACCACTAGTAGGATGATGGTGCCACTCGGGAAC
CTCTCAATTGGGAAGCTGGTATGCTCTCGGGAGCCAAATCACTCGCTTGATAGGTGTGACTCG
GATACCTTACGAACAACTCGGCTAGAGGGTACGTACGATGTGCTGATGACTCGGTTCTTTAAT
AAAAGACGAGGTGCCCTCTAAAGCCTTTTATGGGTCGGATTGTGAGTCCTTCCCAATCACTCT
GCCGACATTAAAATTGACCATGCGAGATTTTTTAGCCTACGAGAGCACTTGGCTGGTAAGATG
GGTTAGCAACATCGGGATTGCGAAGGACTCAGGGGCTGCTAATGATGACACATAACCCGGGTA
TCTACTAGATGGGCTTAGGCCACCACTCGGATAGGGGGTGGGCCGGGCCGCCAGGTCAAGACC
AGCTGCTATACGACTCGGAATAACACTGCCATGTGCCCCAAGGTGTCGGAAGTCCAAGATTAG
CTAGGCTTTCTATAAACCTGTTGTAATACGATACGAAAGATAGGTGCCGACTTGGCTCAGGAC
TCCGATTGTAATCCTGCCTCTTAGACTATATAACTAGGGGCAGGGTACCCCCTTCAAACATTC
AATCTCGTCTCCAGCATAAGAAAACAATAGCCACCAAAGCAGGACATAGGGTATTACGCCATT
GGCGGCCTGAACCTGTCTAAATCGTGTCTCTATGATTTTCCTCGACAACCCTACATATAAATC
TACTCGTCTGCACAAAGCTATGACAACTATATATTGTAAAATTACTTTCATGAAGCCCCAAGT
AACATAAACTTTGTATTGTCAATGTATGTAACTATTTATATATCGATGATATTCACATGTAAC
TAATTTTATGGTCAATGTTAAAAAAAATTGAATCAGTAAACAACTTATATTTCAGATTGGATG
GAGTATGAAACAGTGGTTGAAAATATCTCAAGCACTGATTGAAGCATGTACGACAAAGCTTCC
GATTGCAATAATGAGAACATATAGGTGGCATCTCGGCTTTCTACGTGGACTAATGACAAAGAG
AAGATGCACATCATGCCGCATATAATTCAATGGACTAATGATGGTTTATCGGCTCAAGCATTA
ACAATAGACATCAGTGGATTGGGAAGGCAGACCCAGCATCTTCTTTCATGAGATAGAACACGC
AATGGTGACCCAAAGTAAGTATATTCTCTCTCGATTTCTTCCTTTCATTCCAGGTCCAACCAA
ATCATGATGTTGTAGGATGATGTGTTACACCATCAGAACAAGAAGATCTTTCTTTTTTTTTTT
CTCGAGCGAGGTTAGAGCACATACAAGGTATAATATCAGCTGCCTGTAAGAGTTGCCACGTAG
GATTTCTAGCGATGCAAAGAGGTTATAATGAGGAGAGAGATTGTTGTCTCTTGACGAAATGAC
TATCTATAATATAAGAAAAGAGTCGAGATGTGTCTTGTAGGATATCTACTATGAGGCAACAAC
AATAGATTGTGGGTTATACAAGCTGTCATTTGAATTGTCTAAAAATGACATGTACAGTGTATA
GACAGCAGCCATTTTCAACTGTTGTATGTATCCTTACAGAATGATAGAAGTAAAACTCTCATG
CAACAAATGCTTGACAAGGGCTCTAGAGTTTACGCGGTGTGCATGCCTAGACTCAACTCATAA
GCTACCCTATTTGGTCTTGCCATGTAAATTAAATACGGTAATTATTATCTTGGGATACATGTC
AGAATATGATGTAATTATTACTTGTTCAAAAGAATATCTTCAATTTGGCATTGTCTTTTTCGT
TCGAACTAACTCTGACTGTTAATGAACTATCAATACATACTGACAGGATGTACCTATCTTTCT
TGATCGGAAGAgATGGACAGTTAACCTTGAATATATTTGGACGACACATACATTGCATTATTC
TTTTTGACGGTTGGGAGAAGCCGTACCATACCGAATAGCCATGAAATATGTTAGGTTCCTAGC
ACGGTTTTAAAATTCTAATGTCGTTTTGGCTGTCCCTCAATAAACCCAGCTACTCGGTGATAA
TCTGAAAGCGTTACATTGACACATGAATAAGACAGCACACCACATTGCATTATTCTTTTTGAC
GGCTGGGAGAAGCCATACCATACCGAATAGCCATGAAATATGTCAGATTCGTAGCACGGTTTT
AAAATTCTAATATCGTTTTGGCTGTCCCTCAATAAACCCAGCTACTCGGTGACATTCTGAAAG
CATTACATTGACACATGAATGAGACAGCACACCACATTGCATTATTCTTTTTGACGGCTGGGA
GAAGCCATACCATACCGAATAGCCATGAAATATGTTAGATTCCTAGCACGGTTTTAAAATTCT
AATACTCCCTCCGTTCATTTATCTCCATCATTTTAGCCTTCGGCGTAGTGACCAAGGAGCAGA
GTAAAACCACTCAGTTTGCATTTAATCATTGCGTGCTGAGCGCATACGAGGAGTCACAGGTTA
GGAACAACCAATGAACAGTAAAGAGACAATAAATGCAAACGGGTGGTAGCCAATGAGCAGCAA
ACAGAAAATAAATGCGGGCAAGTCCCAAGCTAATATGAAGGAGATTTTTGAATAAAAAGTTTT
TGCTTAGATGAAGGAGATAAATAAATGGAGGGAATATCGTTTTAGCTGTCCCTCAATAAACCC
AACTACTCGGTGACAATCCGAAAGCGTTATATTGACACATGACTGAGACAGCACACCATCCTC
TTTCCCCTATCGCTTGGCTCCATCTCCATCACTTCCCAGGGGCCAGGGGCCAAATAGGAGCGG
GCAAATAATGGACAAAAATTACTTCTTTAATCGAATGCCCACCCTGCCAAATTCCAAGGATTC
CTTCCTCCAGATATAAGAGTCATCGTCCATGGGTTTCTTGGCCGCAGCTCCTTCAGTTGGCTC
TTGGTCGGCTTCTTGCCCATTACTGCTCTTCCTGAGAGAACCTCGAATTCCGCAAGAATTTAA
CTCGCTGTTGCAGGGATCGTTGAGCTCACGTGCAGGGGGAAAAC (SEQ ID NO: 5)

In some aspects, a construct herein includes a first Saccharum TST promotor. In some aspects, a construct herein includes a first Saccharum TST promotor and a second Saccharum TST promotor. In some aspects, the nucleotide sequence of the first Saccharum TST promoter and second Saccharum TST promoter are the same. In some aspects, the nucleotide sequence of the first Saccharum TST promoter and second Saccharum TST promoter are different. In some aspects, a Saccharum TST promotor is selected from any one of SEQ ID NOs: 1-5. In some aspects, the first Saccharum TST promoter and/or second Saccharum TST promoter has the nucleotide sequence of SEQ ID NO: 3. In some aspects, the first Saccharum TST promoter and/or second Saccharum TST promoter has the nucleotide sequence of SEQ ID NO: 4.

Any number of methods may be used to isolate the promoter sequences disclosed herein, or fragments of the same that retain stem cell-specific promoter activity. The promoter sequences disclosed herein are about 5000 nucleotides in length. As used herein, a “promoter fragment” refers to a promoter that is less than about 5000 nucleotides in length. In some aspects, the promoter fragment is about 100 to about 4900 nucleotides in length, e.g., 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1 175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, 4025, 4050, 4075, 4100, 4125, 4150, 4175, 4200, 4225, 4250, 4275, 4300, 4325, 4350, 4375, 4400, 4425, 4450, 4475, 4500, 4525, 4550, 4575, 4600, 4625, 4650, 4675, 4700, 4725, 4750, 4775, 4800, 4825, 4850, 4875, or 4900 nucleotides. In some aspects, the promoter fragment is a 1000 to 4500 nucleotide fragment of any one of SEQ ID NOs: 1-5, which retains stem cell-specific promoter activity.

In some aspects, the isolation of promoters and promoter fragments may be achieved using a PCR-based approach, e.g., inverse PCR (IPCR), Y-shaped PCR and genome walking approaches. A promoter or promoter fragment of this invention may also be obtained by other techniques such as by directly synthesizing the promoter by chemical means, as is commonly practiced by using an automated oligonucleotide synthesizer. Promoters may also be obtained by application of nucleic acid reproduction technology, such as the PCR (polymerase chain reaction) technology by recombinant DNA techniques generally known to those of skill in the art of molecular biology.

Those of skill in the art are aware of methods for the preparation of plant genomic DNA to obtain a promoter of interest. In one approach, genomic DNA libraries are prepared from a chosen species by partial digestion with a restriction enzyme and size selecting the DNA fragments within a particular size range. The genomic DNA may be cloned into a suitable construct including to but not limited a bacteriophage, and prepared using a suitable construct such as a bacteriophage using a suitable cloning kit from any number of vendors (see for example Stratagene, La Jolla, CA, or Gibco BRL, Gaithersburg, MD).

In some aspects, the nucleotide sequences of the promoters disclosed herein may be modified. Those skilled in the art can create DNA molecules that have variations in the nucleotide sequence. The nucleotide sequences of the present invention as shown in SEQ ID NOs: 1-5 may be modified or altered to enhance their control characteristics. For example, the sequences may be modified by insertion, deletion or replacement of template sequences in a PCR-based DNA modification approach. “Variant” DNA molecules are DNA molecules containing changes in which one or more nucleotides of a native sequence is deleted, added, and/or substituted, preferably while substantially maintaining promoter function. In the case of a promoter fragment, “variant” DNA can include changes affecting the transcription of a minimal promoter to which it is operably linked. Variant DNA molecules may be produced, for example, by standard DNA mutagenesis techniques or by chemically synthesizing the variant DNA molecule or a portion thereof. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs known in the art using default parameters. Promoter activity may be measured by using techniques such as Northern blot analysis, reporter activity measurements taken from transcriptional fusions, and the like. Alternatively, levels of a reporter gene such as green fluorescent protein (GFP) or yellow fluorescent protein (YFP) or the like produced under the control of a promoter fragment or variant can be measured.

To facilitate sugar flux toward fatty acid and/or oil production in cells or tissues (e.g., stem cells or stem tissues) of a plant, some aspects of a construct herein provide for nucleic acids encoding a plastidic phosphoenolpyruvate/phosphate translocator (PPT) protein operably linked to the first Saccharum TST promotor. In some aspects, a construct herein further includes nucleic acids encoding a tonoplast-localized hexose transporter (SWEET16) protein operably linked to the second Saccharum TST promotor. A first nucleic acid sequence is “operably linked” with a second nucleic acid sequence when the sequences are so arranged that the first nucleic acid sequence affects the function of the second nucleic-acid sequence. In some aspects, the first and second nucleic acid sequences are part of a single contiguous nucleic acid molecule and more preferably are adjacent. For example, a promoter is operably linked to a coding sequence if the promoter regulates or mediates transcription of the coding sequence in a cell.

In some aspects, a PPT and/or hexose transporter may be exogenous to a plant or cell in which it is expressed. As used herein, the term “exogenous” refers to a substance coming from some source other than its native source or location. For example, the terms “exogenous protein,” or “exogenous gene” or “exogenous nucleic acid” refer to a protein or nucleic acid from a non-native source, and that has been artificially supplied to a biological system.

“Plastidic phosphoenolpyruvate/phosphate translocator” or “PPT” refers to an inner envelope membrane protein, the function of which to is transport phosphoenolpyruvate (PEP) from the cytosol into plastids for fatty acid and other metabolite biosynthesis. In some aspects, the PPT is a plastid PPT protein. In some aspects, the PPT is a chloroplastic PPT protein. In some aspects, the PPT has an amino acid sequence of any one of the PPT proteins listed in Table 2.

TABLE 2

| Source | GENBANK Accession No. |
|---|---|
| Sorghum bicolor | XP_002454816.1, KAG0535586.1 |
| Miscanthus lutarioriparius | CAD6241577.1 |
| Saccharum hybrid cultivar | QHD26885.1 |
| Zea mays | NP_001150021.1 |
| Setaria italica | XP_004973249.1 |
| Hordeum vulgare | XP_044984343.1 |
| Arabidopsis thaliana | NP_566142.1 |

In some aspects, the PPT is isolated from Sorghum bicolor and has the amino acid sequence:

(SEQ ID NO: 6)
MQSAAAFRPCPAGAGAGAGQLVSRNPSRGPLLPVPARPLRVVVSAATTR
ALGLGRLRLSASPDDRSGQRQVSCGAAGDAVAAPSAEEGGGFMKTLWLG
SLFGLWYLFNIYFNIYNKQVLKVFPYPINITEAQFAVGSVVSLFFWTTG
IIKRPKISGAQLAAILPLAIVHTMGNLFTNMSLGKVAVSFTHTIKAMEP
FFSVLLSAIFLGEFPTVWVVASLLPIVGGVALASLTEASFNWIGFWSAM
ASNVTFQSRNVLSKKLMVKKEESLDNLNLFSIITVMSFFVLAPVTFFTE
GVKITPTFLQSAGLNVNQVLTRSLLAGLCFHAYQQVSYMILAMVSPVTH
SVGNCVKRVVVIVTSVLFFRTPVSPINSLGTAIALAGVFLYSQLKRLKP
KPKTP.

“Tonoplast-localized hexose transporter,” “Sugar Will Eventually be Exported Transporter 16” or “SWEET16” refers to bidirectional vacuolar hexose transport protein that exports stored sugars from the vacuole to the cytosol without leading to too much sugar in the cytosol to inhibit sugar transport from leaves to stems. In some aspects, a hexose transporter has an amino acid sequence of any one of the hexose transporter proteins listed in Table 3.

TABLE 3

| Source | GENBANK Accession No. |
|---|---|
| Sorghum bicolor | XP_002467883.1 |
| Miscanthus lutarioriparius | CAD6211125.1 |
| Saccharum spontaneum | AW014217.1, AYV91999.1 |
| Zea mays | NP_001288522.1 |
| Setaria italica | XP_004984436.1 |
| Arabidopsis thaliana | AAQ65151.1 |

In some aspects, a hexose transporter is isolated from Sorghum bicolor and has the amino acid sequence:

(SEQ ID NO: 7)
MTTPSFLVGIAGNVISILVFASPIATFRRIVRNKSTGDFTWLPYVTTLL
STSLWTFYGLLKPKGLLVVTVNGAGAALEAVYVTLYLVYAPRETKAKMG
KLVLAVNVGFLAVVVAVALLALHGGARLDAVGLLCAAITIGMYAAPLGS
MRTVVKTRSVEYMPFSLSFFLFLNGGVWSVYSLLVRDYFIGVPNAVGFV
LGTAQLVLYLAFRNKAAERKDDDDEKEAAAAAPSSGDEEEGLAHLMGPP
QVEMEMTAQQRGRLRLHKGQSLPKPPTGGPLSSSSSSSPHHGFGSIIKS
LSATPVELHSVLYQHGLGRGRFEPVKKDDVDATNH.

For the practice of the present invention, conventional compositions and methods for preparing and using DNA constructs and host plant cells may be employed. A number of DNA constructs suitable for stable transfection of plant cells or for the establishment of transgenic plants have been described in, e.g., Pouwels et al., Cloning Vectors: A Laboratory Manual, 1985, supp. 1987; Weissbach & Weissbach, Methods for Plant Molecular Biology, Academic Press, 1989; Gelvin et al., Plant Molecular Biology Manual, Kluwer Academic Publishers, 1990; and R.R.D. Croy Plant Molecular Biology LabFax, BIOS Scientific Publishers, 1993. Plant expression constructs may include, for example, the PPT and hexose transporter described herein under the transcriptional control of the promoters described herein as well as other 5′ and 3′ regulatory sequences (e. g., transcription termination site and a polyadenylation signal). Convenient termination regions are available from the Ti-plasmid of A. tumefaciens, such as the octopine synthase and nopaline synthase termination regions. Constructs can also include a selectable marker as described to select for host cells containing the expression construct. Other sequences of bacterial origin may also be included to allow the construct to be cloned in a bacterial host. A construct may also typically contain a broad host range prokaryotic origin of replication. In some aspects, the host cell is a plant cell and the plant expression construct comprises a first promoter as disclosed in any one of SEQ ID NOs: 1-5, an operably linked transcribable nucleotide sequence encoding PPT, and a transcription termination sequence; and optionally a second promoter as disclosed in any one of SEQ ID NOs: 1-5, an operably linked transcribable nucleotide sequence encoding a hexose transporter, and a transcription termination sequence. Other regulatory sequences envisioned as genetic components in an expression construct include but are not limited to non-translated leader sequence which can be coupled with the promoter. Plant expression constructs also can include additional sequences such as polylinker sequences that contain restriction enzyme sites that are useful for cloning purposes.

In some aspects, a construct herein may further include a nucleotide sequence encoding a selectable, screenable, or scorable marker. In some aspects, the marker functions in a regenerable plant tissue to produce a compound which would confer upon the plant tissue resistance to an otherwise toxic compound. Other selectable, screenable, or scorable markers include, but are not limited to, GUS (coding sequence for beta-glucuronidase), GFP (coding sequence for green fluorescent protein), LUX (coding gene for luciferase), antibiotic resistance marker genes, or herbicide tolerance genes. Examples of transposons and associated antibiotic resistance genes include the transposons Tns (bla), Tn5 (nptII), Tn7 (dhfr), penicillins, kanamycin (and neomycin, G418, bleomycin); methotrexate (and trimethoprim); chloramphenicol; kanamycin and tetracycline.

A promoter disclosed herein, as well as variants and fragments thereof, is useful for genetic engineering of plants, e.g., to produce a transformed or transgenic plant, to express a phenotype of interest, e.g., increased fatty acid and/or oil accumulation in stems. Accordingly, the present invention also provides a method for producing a genetically modified plant or plant cell having increased fatty acid and/or oil accumulation in stem cells by transforming one or more host plants or plant cells with a construct of this invention; and selecting one or more transformed plants or plant cells for increased fatty acid and/or oil accumulation in stem cells as compared to a control plant or plant cell lacking the construct, e.g., at least about a 5%, 10%, 15%, 20%, 25, 30%, 35%, 40%, 45%, 50%, or more increase in fatty acid accumulation in stem cells and/or at least about a 5%, 10%, 158, 20%, 25, 30%, 35%, 40%, 45%, 50%, or more increase in oil accumulation in stem cells. In some aspects, increases in fatty acid and/or oil accumulation are determined on a dry weight and/or fresh weight basis. In some aspects, the method further includes the step of regenerating a plant from the selected plant cell comprising the construct. In some aspects, the host plant or plant cell is sugarcane or energycane. In some aspects, one or more host plants or plant cells are transformed with a construct comprising a first Saccharum TST promotor operably linked to nucleic acids encoding an exogenous PPT protein, e.g., a PPT protein having an amino acid sequence of SEQ ID NO: 6; and a second Saccharum TST promotor operably linked to nucleic acids encoding an exogenous tonoplast-localized hexose transporter protein, e.g., a tonoplast-localized hexose transporter protein has an amino acid sequence of SEQ ID NO: 7. In some aspects, the first Saccharum TST promoter and second Saccharum TST promoter are different, and optionally selected from a promoter comprising a nucleotide sequence of any one of SEQ ID NOS: 1-5.

Also provided herein is a method for producing a genetically modified plant having increased fatty acid and/or oil accumulation by expressing an exogenous PPT protein in a tissue of a plant. In some aspects, a tissue of a plant expressing an exogenous PPT protein exhibits at least about a 5%, 10%, 15%, 20%, 25, 30%, 35%, 40%, 45%, 50%, or more increase in fatty acid accumulation and/or at least about a 5%, 10%, 15%, 20%, 25, 30%, 35%, 40%, 45%, 50%, or more increase in oil accumulation in the tissue expressing the PPT protein as compared to the same tissue in a plant that has not been genetically modified to express an exogenous PPT protein and grown under the same conditions. In some aspects, the plant is sugarcane or energycane. In some aspect, a genetically modified plant or plant cell is produced by transforming the plant or plant cell with a construct comprising nucleic acids encoding an exogenous PPT protein operably linked to a promoter and, in the case of a plant cell, regenerating a plant. In some aspects, expression of the PPT protein is mediated by a Saccharum TST promoter comprising a nucleotide sequence selected from the group of SEQ ID NOs: 1-5. In some aspects, the exogenous PPT protein has an amino acid sequence of SEQ ID NO: 6.

In some aspects, an exogenous PPT protein is expressed in a specific plant tissue. In some aspects, an exogenous PPT protein is expressed in a plant tissue that accumulates sugar, e.g., soluble sugars such as glucose, fructose and/or sucrose. A “tissue that accumulates sugar” or “sugar-rich tissue” refers to a tissue having at least about a 2-fold (e.g., about a 2-fold, 3-fold, 4-fold, 5-fold, 6-fold, 7-fold, 8-fold, 9-fold or more) or higher level of soluble sugar (e.g., on a dry weight basis) compared to another tissue of the plant. In some aspects, a tissue that accumulates sugar may naturally accumulate sugar, e.g., a sink tissue such as a meristem, root, flower, or fruit. In some aspects, a tissue that accumulates sugar may be genetically engineered to accumulate sugar. In some aspects, knockout of one or more sugar transporters of the SWEET family may be used to generate leaf tissue that accumulates sugar. In some aspects, tissue-specific expression (e.g., overexpression) of one or more members of the SWEET family provides a tissue that accumulates sugar. In some aspects, stem cell-specific expression of a SWEET protein may be used to generate stem tissue that accumulates sugar. In some aspects, the plant is sugarcane or energycane and cells of the tissue that accumulate sugar express an exogenous tonoplast-localized hexose transporter protein, e.g., tonoplast-localized hexose transporter protein has an amino acid sequence of SEQ ID NO: 7.

As used herein, a “fatty acid” refers to aliphatic acids (alkanoic acids) of varying chain length, typically from about 4 to 22 carbon atoms in length.

An “oil” refers to a lipid substance that is liquid at 25° C. and usually polyunsaturated. In some aspects, an oil is referred to as a triacylglycerol. A “triacylglycerol(s)” or its abbreviation “TAG(s),” also known as “triglyceride(s)” or “TG” or “oil,” refer to neutral lipids composed of three fatty acyl residues esterified to a glycerol molecule (and such terms may be used interchangeably throughout the present disclosure). Such oils may contain long chain PUFAs, as well as shorter saturated and unsaturated fatty acids and longer chain saturated fatty acids. Thus, “oil biosynthesis,” “oil synthesis,” or “oil accumulation” generically refers to the synthesis of TAGs in the cell.

In some aspects of the methods herein, an increase in fatty acid and/or oil accumulation is determined in a mature plant. “Mature plant” refers to a plant at any desired developmental stage beyond that of the seedling. Seedling refers to a young immature plant at an early developmental stage. In some aspects, a mature plant is a plant at a developmental stage when the plant or a part thereof (e.g., seed, fruit, flower, stem, and/or leaf) is ready for harvest.

As used herein, the terms “transforming” or “introducing” means presenting the construct of interest to the plant, plant part, or plant tissue in such a manner that the construct gains access to the interior of a cell. Where more than one polynucleotide of interest is to be introduced, these polynucleotides may be assembled as part of a single polynucleotide or DNA construct, as or separate polynucleotides or DNA constructs, and can be located on the same or different transformation vectors. Accordingly, these polynucleotides may be introduced into plant cells (e.g., sugarcane or energycane) in a single transformation event, in separate transformation events, or, e.g., as part of a breeding protocol.

A “transformed plant” and “transgenic plant” refer to a plant that comprises within its genome a heterologous polynucleotide. “Heterologous” polynucleotides refer to polynucleotides that originate from a foreign source or species or, if from the same source, is modified from its original form. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression construct that includes nucleic acids of interest, the regeneration of a population of plants resulting from the insertion of the transferred nucleic acids into the genome of the plant and selection of a plant characterized by insertion into a particular genome location. An event is characterized phenotypically by the expression of the inserted nucleic acids. At the genetic level, an event is part of the genetic makeup of a plant. The term “event” also refers to progeny produced by a sexual cross between the transformant and another plant wherein the progeny include the heterologous DNA.

Transformation protocols as well as protocols for introducing DNA constructs into plants may vary depending on the type of plant or plant cell, i.e., monocot or dicot, targeted for transformation. Suitable methods of introducing DNA constructs into plant cells and subsequent insertion into the plant genome include microinjection, electroporation, Agrobacterium-mediated transformation, direct gene transfer, and ballistic particle acceleration. Methods and compositions for rapid plant transformation are also found in US 2017/0121722, herein incorporated by reference in its entirety. The construct of this invention may also be introduced into the genome of a plant cell using double-stranded break technologies such as TALENS, meganucleases, zinc finger nucleases, CRISPR-Cas, and the like.

In some aspects, method of this invention further includes the step of regenerating a plant from a plant cell. As used herein, “regenerate,” “regeneration,” and “regenerating” (and grammatical variations thereof) means formation of a plant from various plant parts (e.g., plant explants, callus tissue, plant cells) that includes a rooted shoot. The regeneration of plants from various plant parts is well known in the art. See, e.g., Methods for Plant Molecular Biology (Weissbach et al., eds. Academic Press, Inc. (1988); for regeneration of sugarcane plants, see, for example, Arencibia et al. (Trans. Res. 7:213-222 (1998)); Elliot et al. (Plant Cell Rep. 18:707-714 (1999)); and Enriquez-Obregon et al. (Planta 206:20-27 (1998)). For example, regenerating plants containing a construct introduced by Agrobacterium from leaf explants can be achieved as described by Horsch et al. (1985) Science 227:1229-1231. Briefly, transformants are grown in the presence of a selection agent and in a medium that induces the regeneration of shoots (e.g., in sugarcane). See, for example, Fraley et al. (1983) Proc. Natl. Acad. Sci. USA 80:4803-4807. This method typically produces shoots within about two weeks to four weeks, and the transformed shoots are then transferred to an appropriate root-inducing medium containing the selective agent and an antibiotic to prevent further bacterial growth. Typically, transformed shoots that root in the presence of the selective agent to form plantlets are then transplanted to soil or other media to allow the production of additional roots. For references to regeneration of sugarcane, see Lakshmann et al. In Vitro Cell Devel Biol 41:345-363 (2005).

The transgenic plantlets are then propagated in soil or a soil substitute to promote growth into a mature transgenic plant. Propagation of transgenic plants from these plantlets is performed, e.g., in perlite, peatmoss and sand (1:1:1) or commercial plant potting mix under glasshouse conditions. These plants may then be grown, and either pollinated with the same transformed strain or different strains, and the resulting progeny having expression of the desired phenotypic characteristic. Two or more generations may be grown to ensure that expression of the desired phenotypic characteristic is stably maintained and inherited and then seeds harvested to ensure expression of the desired phenotypic characteristic has been achieved, in this manner, the present disclosure provides transformed seed (also referred to as “transgenic seed”) having a DNA construct of the disclosure stably incorporated into its genome.

The present disclosure also includes transgenic plants obtained by any of the disclosed methods or using any of the disclosed constructs herein. The present disclosure also includes seeds from a plant obtained by any of the disclosed methods or compositions herein. Suitable plants or plant cells include, but are not limited, alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, energycane, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yarns, or zucchini. In some aspects, the transgenic plant is Arabidopsis, corn, wheat, soybean, or cotton. In some aspects, the transgenic plant is sugarcane, energycane, sorghum or miscanthus, in which stems are the major carbohydrate (e.g., soluble sugar) sink tissues.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

The following examples are provided for illustrative purposes only and not intended to limit the scope of the claims.

### EXAMPLE 1: MATERIALS AND METHODS

Plant materials and growth conditions. The Arabidopsis (Arabidopsis thaliana (L.) Heynh.) plants were grown under a controlled condition with a temperature of 22° C., the light intensity of 100˜150 μmol m−2 s−1, and a 16-h light/8-h dark photoperiod. T-DNA mutants of atsweet13 (SALK_087791), were obtained from ABRC. atsweet11;12 double mutants as described (Chen et al. (2012) Science 335:207-211). Homozygous atsweet11;12;13 mutants were created via crossing and were genotyped using primers and used in related experiments. The floral dip transformation method (Clough et al. (1998) Plant J. 16:735-743) was used to generate all the transgenic lines described herein. At least 16 T1 lines were generated for each construct, and at least 3 transgene-positive T2 lines with strong fluorescence signal were used in relevant experiments. All plants were cultivated in pots containing potting mix (PRO-MIX, Premier tech home and garden, Québec, 414 Canada). All Arabidopsis leaf samples were collected at 21 days post germination (DPG) unless otherwise specified.

Sugarcane cultivar CP88-1762 and transgenic sugarcane plants were maintained in the Plant Biology greenhouse at the University of Illinois at Urbana-Champaign. Plants were grown under controlled temperature (28° C./22° C., day/night) with a 14-h light (approximately 300-950 μmol m−2 s−1)/10-h dark regimen provided by a mix of natural photoperiod and light conditions. Transgenic sugarcanes (and WT) were propagated by nodal stem cuttings to obtain at least 3 biological replicates per line and were each planted in potting mix (BM6, Berger, Québec, Canada).

Constructs for Arabidopsis transformation. The coding sequence region of AtPPT1 (At5G33320) was synthesized (GenScript, NJ, USA) with synonym mutations made to remove internal DraIII, BbsI, and BsaI restriction sites, and was subcloned into pLOV to make pL0M-SC1-AtPPT1. Subsequently, pL1M-F2-p35S-AtPPT1-eGFP-tNOS was assembled using the Golden Gate cloning protocol (Weber et al. (2011) PLoS ONE 6:e16765) before further assembling into a binary vector (pL2V-1) with pL1M-R1-p35S-HYG-tNOS. The binary vector was incorporated into Agrobacterium tumefaciens strain GV3101 before transforming into wild-type col-0 and atsweet11;12;13.

Constructs for sugarcane transformation. The sugarcane/energycane stem-specific promoters (pTST2b1A-CP88, pTST2b1A-UFCP82) and the coding region of SbPPT1(Sobic.004G353100), SbSWEET16 (Sobic. 001G377600) were synthesized (GenScript, NJ, USA) with internal DraIII, BbsI, and BsaI restriction sites removed, and were subcloned into pL0V to make their respective level-0 modules. Subsequently, pL1a-pTST2b1A-mCP88-PPT1-tPvUbiII and pL1b-pTST2b1A-mUFCP82-SWEET16-tPvUbiII were assembled using the Golden Gate cloning protocol. Together with the selectable marker module (pL1c-pZmUbi-NPTII-tSbHSP), three level-1 modules were assembled into a level-2 module, followed by digestion using AscI to remove the backbone; the gel purified fragment was used for biolistic transformation.

Sugarcane transformation. Biolistic gene transfer into embryogenic callus derived from immature leaf-whorl cross-sections was used for sugarcane transformation as described earlier (Taparia et al. (2012) Plant Cell Tiss. Organ Cult. 111:131-141).

Fatty acids determination. The total fatty acid profiles from ˜20 mg freeze-dried samples were determined using a direct acid-catalyzed transmethylation method, as previously reported (Wang et al. (2022) New Phytologist 236:525-537).

Soluble sugar determination. Soluble sugar components from ˜5 mg freeze-dried samples were determined as previously reported (Wang et al. (2022) New Phytologist 236:525-537) using a high-performance liquid chromatography (HPLC) coupled with a refractive index detector (RID).

Nile Red staining. Lipid droplets from Arabidopsis leaves were visualized via Nile Red staining as previously reported (Winichayakul et al. (2013) Plant Physiol. 162:626-639) using confocal microscopy.

Microscopy imaging. For GFP acquisition, images were taken using LSM 880 (Carl Zeiss, NY, USA). Argon laser excitation wavelength and emission bandwidths were 488 nm (10% intensity) and 493-545 nm for GFP (gain 600), 633 nm (10% intensity) and 634-686 nm for chlorophyll autofluorescence (gain 600), respectively. For Nile Red acquisition, images were taken using LSM 710 (Carl Zeiss, NY, USA). Argon laser excitation wavelength and emission bandwidths were 514 nm (10% intensity) and 557-599 nm for Nile Red (gain 800), 514 nm (10% intensity), and 619-697 nm for chlorophyll autofluorescence (gain 350), respectively.

RNA isolation and RT-qPCR. RNA isolation and RT-qPCR was performed as previously described (Wang et al. (2021) GCB Bioenergy 13:1515-1527). The expression values were normalized to ACT8 for Arabidopsis or GAPDH for sugarcane in each repeat (Livak et al. (2001) Methods 25:402-408). The analyses were based on 3 biological replicates.

RNA-seq analysis. The cDNA libraries were prepared from 1 μg RNA and were sequenced at the Roy J. Carver Biotechnology Center at the University of Illinois at Urbana-Champaign. Fastq files were trimmed using Trimmomatic (Bolger et al. (2014) Bioinformatics 30:2114-2120) with a minimum length of 60. They were aligned to the Araport11 gene set (Cheng et al. (2017) Plant J. 89:789-804) with the STAR aligner program (Dobin et al. (2013) Bioinformatics 29:15-21) to produce BAM files, and these were converted to counts with the featcounts program (Liao et al. (2014) Bioinformatics 30:923-930).

Differentially Expressed Gene (DEG) analysis was carried out in R studio using the DESeq2 package from Bioconductor with an adjusted p-value cutoff of 0.01 and a log2 fold change cutoff of 1 (Gentleman et al. (2004) Genome Biol. 5:R80; Love et al. (2014) Genome Biol. 15:550). Principal Component Analyses (PCA) were carried out on a log transformation of the count data using the plotPCA function of DESeq2 and ggplot2. For all results tables, only genes whose median expression in at least one treatment group was 5 TPM or greater were included. Heatmaps were carried out on TPM data converted to z-scores for each gene using the pheatmap package. Heatmaps and gene ontology (GO) were performed using the OmicShare tools.

LC/MS and GC/MS analysis. Metabolite analysis was performed by the Metabolomics Core Facility of the Roy J. Carver Biotechnology Center, University of Illinois Urbana-Champaign. Samples were homogenized with Bead Mill 4 (Fischer Scientific, MA, USA) and compounds were partitioned in 1 mL of methanol:chloroform (2:3 v/v) (lipid analysis) and 1 mL of distilled water (glycolysis intermediates analysis) followed by centrifuging at 14,000 rpm for 15 minutes. Supernatants were collected into separate tubes, evaporated and resuspended in 100 μL of methanol: chloroform (2:3 v/v) or 20 mM ammonia acetate.

PEP, DHAP, G3P, FBP, 2PGA, 3PGA and Acetyl-CoA were analyzed using LC-MS. Subsequently, 10 μL of each ammonia acetate dissolved sample was injected into on Agilent 1200 system (Agilent, CA, USA) and the liquid chromatography separation was performed on a Kinetex C18 100A column (Phenomenex, CA, USA) with mobile phase A (20 mM ammonia acetate in water) and mobile phase B (methanol). Mass spectra were acquired with SCIEX 5500 mass spectrometer under positive and negative electrospray ionization with the ion spray voltage at +5500 V. Peak integration and quantitation were performed using Analyst 1.7.1 software. Calibration curves were built for the 0.1-50 μM range.

F6P, G6P and Pyr were analyzed using GC-MS. Subsequently, 100 μL of each sample was dried under vacuum and derivatized with 50 μL methoxyamine hydrochloride (Sigma-Aldrich, MO, USA) (40 mg/ml in pyridine) for 60 minutes at 50° C., then with 50 μL MSTFA+1% TMCS (Thermo, MA, USA) at 70° C. for 120 minutes, following a 2-hour incubation at room temperature. Chromatograms were acquired using a GC-MS system (Agilent Inc, CA, USA) consisting of an Agilent 7890 gas chromatograph, an Agilent 5975 MSD and a HP 7683B autosampler. Gas chromatography was performed on a ZB-5MS capillary column (Phenomenex, CA, USA). The inlet and MS interface temperatures were 250° C., and the ion source temperature was adjusted to 230° C. An aliquot of 1 μL was injected in a splitless mode. The temperature program was: 5-min isothermal heating at 70° C., followed by an oven temperature increase of 5° C. min⁻¹ to 310° C. and a final 10 min at 310° C. The mass spectrometer was operated in positive electron impact mode (EI) at 69.9 eV ionization energy at m/z 30-800 scan range. (13)C6 Glucose spiked with was samples prior to derivatization and was used as an internal standard. Target peaks were evaluated by the Mass Hunter Quantitative Analysis B.08.00 (Agilent, CA, USA) software. Calibration curve was built for 0.1-50 μg/mL concentration range.

Untargeted lipid profiling. For lipidomic analysis, samples were initially spiked with a mixture of labeled surrogate internal standards. Post-processing extracts were analyzed using the Thermo Q-Exactive mass spectrometer (MS) system (Bremen, Germany), as previously described (Xue et al. (2020) Biotechnol. Bioeng. 117:2131-2138). The Dionex UltiMate 3000 series HPLC system (Thermo, Germering, Germany) was used, with LC separation performed on a Thermo Accucore C18 column with mobile phase A (60% acetonitrile: 40% water with 10 mM ammonium formate and 0.1% formic acid) and mobile phase B (90% isopropanol: 10% acetonitrile with 10 mM ammonium formate and 0.1% formic acid). The injection volume was 10 μl. Mass spectra were acquired under both positive and negative electrospray ionization. Full scan mass spectrum resolution was set to 70,000 with a scan range of m/z 230 to 1,600. For MS/MS scan, the mass spectrum resolution was set to 17,500. All the LC-MS raw data files were performed using MS-DIAL ver.4.90 for software data collection, peak detection, alignment, adduct, and identification (Tsugawa et al. (2015) Nat. Methods 12:523-526). Compounds were annotated by m/z and MS/MS spectra against the LipidBlast mass spectra database (Kind et al. (2013) Nat. Methods 10:755-758).

Statistical analysis. The Shapiro-Wilk test was used to test the normality of data. Reciprocal transformation was applied for data that failed to conform normality. The differences between the two groups were determined using the two-tailed Student's t-test with equal variance. The differences among multiple groups were assessed using one-way analysis of variance (ANOVA) followed by multiple comparison tests (Fisher's least significant difference (LSD) method). ANOVA statistical analysis was performed using Origin 2022 statistical software (OriginLab Corporation, MA, USA). Pearson's correlation coefficients were calculated using SPSS (SPSS Product and Service Solution, NY, USA).

### EXAMPLE 2: GENERATION OF SUGAR-RICH MUTANT SWEET11;12;13

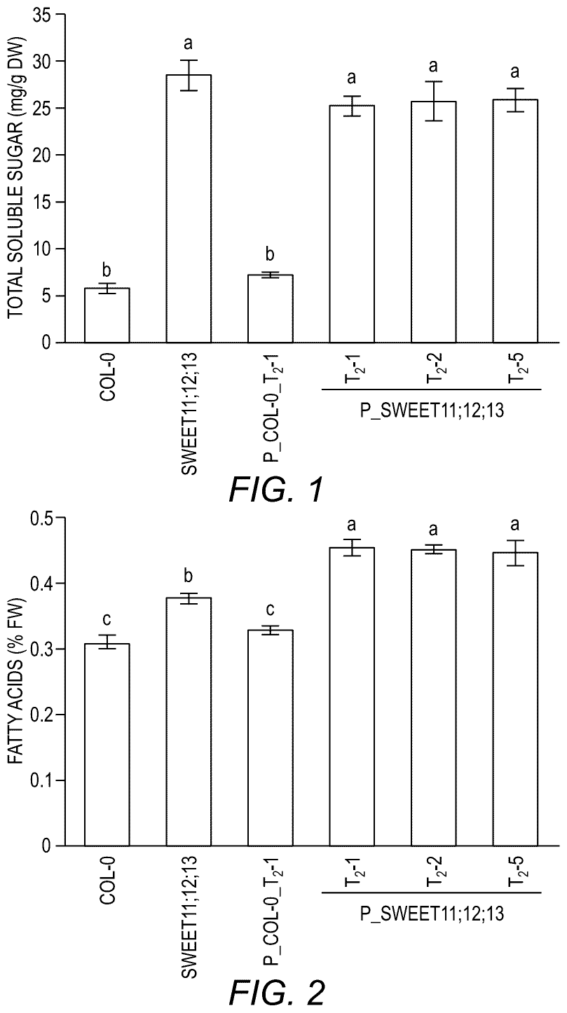

A sugar-rich mutant in the model plant Arabidopsis was generated. Two SWEETs (Sugars Will Eventually be Exported Transporters; Chen et al. (2010) Nature 468:527-532), SWEET11 and SWEET12, are responsible for sugar export from phloem parenchyma cells into apoplasmic space during phloem loading, and their mutant (sweet11;12) accumulates about two-fold of soluble sugars in leaves compared to Col-0 wild-type (Chen et al. (2012) 335:207-211). Interestingly, SWEET13 was highly up-regulated (˜15 fold) in sweet11;12 (Chen et al. (2012) 335:207-211), indicating functional redundancy. To have a mutant with significant sugar accumulation in the leaves, sweet11;12;13 was created by crossing sweet13 T-DNA mutant with sweet11;12 for this study. Morphologically, sweet11;12;13 is stunted compared to Col-0, consistent with the roles of these proteins during phloem loading (Chen et al. (2012) 335:207-211). Indeed, sweet11;12;13 accumulated more than about 5-fold the total soluble sugar when compared to Col-0 (FIG. 1).

### EXAMPLE 3: OVEREXPRESSING PPT1 RESULTED IN AN INCREASE IN FATTY ACID CONTENT IN SUGAR-RICH LEAVES

To evaluate the effects of PPT1 on fatty acid accumulation in sugar-rich sweet11;12;13 mutant, p35S:PPT1-eGFP was transformed into homozygous sweet11;12;13 mutants and Col-0. T1 and T2 transgenic lines were screened using confocal microscopy. Three representative and independent T2 transgenic lines were selected for further analysis. As expected, in all transgenic lines, PPT1 was localized to the periphery enclosing chloroplasts, which produce red autofluorescence, in agreement with its reported chloroplast localization (Knappe et al. (2003) Plant J. 36:411-420). To further confirm the overexpression, RT-qPCR analysis was performed. The results showed that all three independent transgenic lines in both backgrounds had significantly higher PPT1 transcript levels compared to Col-0 with no significant difference between sweet11;12;13 and Col-0. To evaluate the potential phenotypical effects of PPT1 overexpression on vegetative growth, their growth was compared at 21 days post-germination (DPG). Overexpressing PPT1 in either background did not lead to any morphological differences when compared to their respective controls.

Soluble sugar content (including sucrose, glucose and fructose) was analyzed using HPLC, and no significant differences were observed among 3 independent PPT1 overexpression lines in the Col-0 background. Thus, the T2-1 of P_Col-0 (representing overexpressing PPT1 in Col-0 background) was used for the subsequent experiments. The sweet11;12;13 mutant accumulated about 5-fold more total soluble sugar than Col-0, and no significant differences were observed between PPT1 overexpression lines and its control (FIG. 1), although P_sweet11;12;13 (representing overexpressing PPT1 in sweet11;12;13 background) showed slightly reduced sugar levels compared to the sweet11;12;13 mutant. Total fatty acid content was also measured using GC-FID. Although overexpressing PPT1 in Col-0 background had no effect on fatty acid content (FIG. 2), fatty acid levels were significantly higher in sweet11;12;13 compared to Col-0(21.9% increase). Moreover, overexpressing PPT1 in sweet11;12;13 background resulted in a further significant increase in fatty acid compared to sweet11;12;13 (44.5˜46.5% higher than Col-0 and 18.5˜20.1% higher than sweet11;12;13) (FIG. 2). These results demonstrated that overexpressing PPT1 can boost fatty acid biosynthesis specifically in sugar-rich sweet11;12;13 mutant.

### EXAMPLE 4: OVEREXPRESSING PPT1 INCREASED FATTY ACID CONTENT IN SUGAR-RICH OLDER LEAVES OF SWEET11;12;13

Given that sweet11;12;13 is smaller than Col-0 in size, it was determined whether the developmental stage affects the observed increase in fatty acid accumulation due to PPT1 overexpression. Rosette leaf development was carefully monitored until 21 DPG. Both sweet11;12;13 and Col-0 developed a total number of 8 true leaves at 21 DPG, consistent with a well-documented observation for soil-growing Arabidopsis under similar conditions (Boyes et al. (2001) Plant Cell 13:1499-1510). The total of eight true leaves were further categorized into 4 groups: leaves 1&2 (oldest), leaves 3&4, leaves 5&6 and leaves 7&8 (youngest). Total soluble sugar content was determined from these four groups in the Col-0 background. Interestingly, the total sugar levels were slightly higher in leaves 1&2 and leaves 7&8 compared to leaves 3&4 and leaves 5&6. By contrast, sweet11;12;13 displayed a pattern with older leaves progressively accumulate higher sugar content than younger leaves (leaves 1&2>leaves 3&4>leaves 5&6>leaves 7&8), consistent with the roles of SWEET11/12/13 during phloem loading in source leaves (Chen et al. (2012) science 335:207-211). Overexpressing PPT1 did not significantly affect sugar content in either background, although it slightly reduced sugar levels in PPT1_sweet11;12;13, suggesting a fraction of sugar may be converted into other carbon-containing metabolites, such as fatty acids. Interestingly, in older leaf groups (leaves 1&2, leaves 3&4 and leaves 5&6), sweet11;12;13 has significantly higher sugar content than Col-0, while no significant differences were observed between sweet11;12;13 and Col-0 in leaves 7&8. Consistently, sweet11;12;13 accumulated more starch than Col-0 in older leaves (leaves 1-6).

Total fatty acid content was further analyzed in four leaf groups of each plant material. The overall pattern showed fatty acid levels increasing from 1&2<leaves 3&4<leaves 5&6<leaves 7&8 in each plant material. Only in the sugar hyper-accumulating older leaf groups (leaves 1&2 and leaves 3&4), sweet11;12;13 had higher fatty acid levels than Col-0, while overexpressing PPT1 could significantly further boost fatty acid content in sweet11;12;13 background of all three independent lines compared to sweet11;12;13. Such enhanced effect was diminished in leaves 5&6 and disappeared in leaves 7&8. Overexpressing PPT1 in Col-0 background seemed to have a positive role on fatty acid content in leaves 1&2, but not in other leaf groups. These results confirmed that overexpressing PPT1 has a positive effect on fatty acid content under sugar-rich conditions, and such effects are sugar-dependent, regardless of genetic background. Considering leaves 3&4 showed a stark contrast in sugar and fatty acid accumulation, these leaves were used for metabolic and transcriptomic analysis.

### EXAMPLE 5: OVEREXPRESSING PPT1 AFFECTS CARBON FLOW BETWEEN CYTOSOL AND CHLOROPLAST IN THE SUGAR-RICH BACKGROUND

To assess if the increase in fatty acid leads to the accumulation of lipid droplets (LDs), LDs were visualize using confocal microscopy following Nile Red staining. LDs were clearly visible in three P_sweet11;12;13 lines, not in either Col-0 or P_Col-0, while LDs were also presented sporadically in sweet11;12;13. Lipidomic analysis was performed to explore the global lipid changes. The results showed an overall accumulation of lipid species in sweet11;12;13 compared to Col-0 in two distinct vertical clusters. Two horizontal clusters were categorized with cluster 1 lipid species mainly accumulated in P_sweet11;12;13 (T2-2) and cluster 2 lipid species mainly accumulated in sweet11;12;13.

Based on carbon flow from glycolysis to starch, fatty acids, and TAG biosynthesis, intermediate metabolites were quantified using targeted metabolite analysis via LC/MS or GC/MS. Interestingly, all the detected intermediate metabolites (except for pyruvate) were significantly elevated in sweet11;12;13 and their levels were restored to a similar level of Col-0 in P_sweet11;12;13, suggesting the turnover rate of glycolytic intermediates may increase when PPT1 is enhanced in sugar-rich sweet11;12;13 mutants. Additionally, overexpressing PPT1 significantly increased pyruvate content in both Col-0 and sweet11;12;13.

To investigate what genes might regulate these changes, the expression of key enzymes and transporters was analyzed using RT-qPCR. Both glucose 6-phosphate/phosphate translocator (GPT2) and the pyruvate transporter BASS2 were significantly upregulated in three independent lines of P_sweet11;12;13, suggesting the transport of G6P and pyruvate across the plastidic membrane is enhanced in P_sweet11;12;13. Consistent with the increased G6P supply into the plastid, ADP-glucose pyrophosphorylase (ADG1), encoding a key enzyme for starch synthesis, was significantly upregulated in P_sweet11;12;13. Similarly, β-ketoacyl-ACP synthase (KAS1), responsible for fatty acid chain elongation, was also significantly upregulated in P_sweet11;12;13, consistent with increased fatty acid. TAG can be synthesized via either diacylglycerol: acyl-CoA acyltransferase (DGAT) or phospholipid: diacylglycerol acyltransferase (PDAT; Xu & Shanklin (2016) Annu. Rev. Plant Biol. 67:179-206), PDAT1 was significantly upregulated in P_sweet11;12;13 while DGAT1 and DGAT2 remained unaffected. Interestingly, TPS5, encoding a class II trehalose-6P synthase with no apparent enzymatic activity (Ramon et al. (2009) Plant Cell Environ. 32:1015-1032), was significantly upregulated in P_sweet11;12;13. By contrast, the transcription levels of other related genes, including GPT1, triose phosphate/phosphate translocator (TPT), cytosolic enolase (ENOc/LOS2), plastidic pyruvate kinase (PKPβ1), chalcone synthase (TT4), biotin carboxyl carrier protein (BCCP2) a subunit of Acetyl COA-carboxylase (ACCase), and WRI1, were not differentially regulated among the tested materials.

### EXAMPLE 6: OVEREXPRESSING PPT1 ALLEVIATED STRESS RESPONSES IN SWEET11;12;13

To explore other possible interpretations globally, RNA-seq analysis was conducted among different materials. Principal component analysis (PCA) revealed that the three biological replicates of the material with the same background clustered together, with Col-0 and sweet11;12;13 backgrounds clearly distinguished along the PC1 dimension, suggesting impaired phloem loading with enhanced sugar accumulation plays a major role in distinguishing gene expression. Consistent with the genotyping and RT-PCR results, the transcripts of SWEET11, 12 and 13 are significantly reduced in sweet11;12;13.

A heatmap generating both row and column clusters was built. This analysis indicated that the three biological replicates of each material clustered together, forming two major column clusters that separated the Col-0 and sweet11;12;13 background materials. Additionally, four major row clusters were formed: cluster 1 genes were highly expressed in Col-0; cluster 2 genes were highly expressed in both Col-0 and P_Col-0; cluster 3 genes were highly expressed in both sweet11;12;13 and P_sweet11;12;13 in which cluster 3.1 genes exhibited enhanced expression in P_sweet11;12;13; cluster 4 genes were highly expressed in sweet11;12;13 with cluster 4.1 genes peaking in sweet11;12;13 but later decreased substantially in P_sweet11;12;13. Gene Ontology (GO) enrichment analysis revealed that cluster 3 genes were enriched in an array of defense and stress-responsive terms, such as anthocyanin-containing compound biosynthetic process, detoxification, response to hypoxia, jasmonic acid (JA), oxidative stress, fungus, wounding, etc. Cluster 3.1 genes were not only enriched in some of the above-mentioned stress-responsive terms but also enriched with carbohydrate-responsive genes, suggesting the carbon flow was altered when overexpressing PPT1 in sweet11;12;13. Interestingly, cluster 4.1 genes were enriched in several iron ion-responsive terms, including intracellular sequestering of iron, response to iron, multicellular and organismal-level iron homeostasis, indicating the iron responses were activated in sweet11;12;13, but was relieved when overexpressing PPT1.

Among the enriched pathways, several individual genes are of particular interest, in cluster 3.1, the gene expression levels of the chlorophyll A/B binding protein 3 (CAB3), two members of the multidrug and toxic compound extrusion (MATE) family (DTX18 and DXT15), and a vasculature iron transporter (NPF5.8) were upregulated in sweet11;12;13, with further increased expression in P_sweet11;12;13. Additionally, two beta-glucosidases-related genes (BGLU18 and PBP1) were significantly up-regulated in P_sweet11;12;13. In cluster 4.1, the gene expression levels of several key iron transporters/iron binding proteins (VITL2, FER1, FER4, NEET), and genes involved in chloroplast reactive oxygen species (ROS) homeostasis (ENH1, APXS, LSU1) were significantly up-regulated in sweet11;12;13, but in then decreased in P_sweet11;12;13, suggesting overexpressing PPT1 mitigates iron and ROS stress responses under sugar-rich conditions.

### EXAMPLE 7: OVEREXPRESSING SBPPT1 INCREASED FATTY ACID CONTENT AND STIMULATED TAG ACCUMULATION IN TRANSGENIC SUGARCANE

Having demonstrate that overexpressing PPT1 in sugar-rich Arabidopsis mature leaves leads to improved fatty acid and TAG, PPT1 was overexpressed in sugarcane, which hyper-accumulates sugars in stems. Due to the high polyploidy of sugarcane genome, it is challenging to predict which PPT1 allele is functional. Thus, PPT1 from sorghum was selected as sorghum is a diploid close relative species of sugarcane. Four SbPPT homolog genes were identified via a BLAST search in the sorghum genome using Arabidopsis PPT1 protein sequences. A phylogenetic was tree built, including Arabidopsis TPTs, GPTs, PPTs, previously reported BnaPPTs from Brassica napus (Tang et al. (2022) J. Adv. Res. 42:29-40), and SbPPTs. Four SbPPTs were identified and clustered with Arabidopsis and Brassica PPT1, and the closest related gene (Sobic. 004G353100) was designated as SbPPT1 and was later used in the overexpression strategy.

To improve the availability of PEP derived from cytosolic sucrose in the stem, SbSWEET16 was included to reduce the sequestration of sucrose into the vacuole in the engineering strategy. SWEET16, placed in the cluster IV of the SWEET phylogenetic tree (Chen et al. (2010) Nature 468:527-532), encodes a vacuolar sugar transporter, and its overexpression has been shown to enhance freeze-tolerance, likely due to altered cytosolic and vacuolar sugar homeostasis (Klemens et al. (2013) Plant Physiol. 163:1338-1352). Since soluble sugars are predominantly stored in vacuoles within sugarcane stems, overexpressing SWEET16 may accelerate sugar export from vacuoles, thereby increasing sugar availability in the cytosol. For this purpose, both SbPPT1 and SbSWEET16 (Sobic.001G377600) were driven by each of two sugarcane stem-specific TST promoters (Table 4) and the construct was then transformed into sugarcane. RT-qPCR analysis was used to examine the gene expression level of transgenic sugarcane events. The expression data was multiplicative inverse transformed to conform normality for one-way ANOVA analysis. Interestingly, the co-high expression of two genes was not observed as expected. Four independent transgenic lines (PS4, PS8, PS15, PS44) with SbPPT1 significantly overexpressed were used for further analysis, although these transgenic lines showed a trend of increased SbSWEET16 ectopic expression with no significant differences. No obvious growth differences were observed between wild-type CP88-1762 and all four transgenic lines at the mature stage in the greenhouse, suggesting the overall growth was not compromised by PPT1 overexpression.

TABLE 4

| | | Protein encoded by |
|---|---|---|
| Fusion | Promoter | coding sequence |
| pTST2b_1A (mS):: | pTST2b_1A (mS): | PPT: plastidic |
| SbPPT1-tPvUbiII | stem-specific | phosphoenolpyruvate |
| | promoter from | (PEP)/phosphate |
| | sugarcane CP88 with | translocator (SEQ |
| | three ATGs mutated | ID NO: 6) |
| | (SEQ ID NO: 4) | |
| pTST2b_1A (mE):: | pTST2b_1A (mE): | SWEET16: tonoplast- |
| SbSWEET16- | stem-specific | localized hexose |
| tPvUbiII | promoter from | transporter (SEQ ID |
| | energycane UFCP82 | NO: 7) |
| | with three ATGs | |
| | mutated (SEQ ID | |
| | NO: 3) | |

*TST = Tonoplast Sugar Transporter

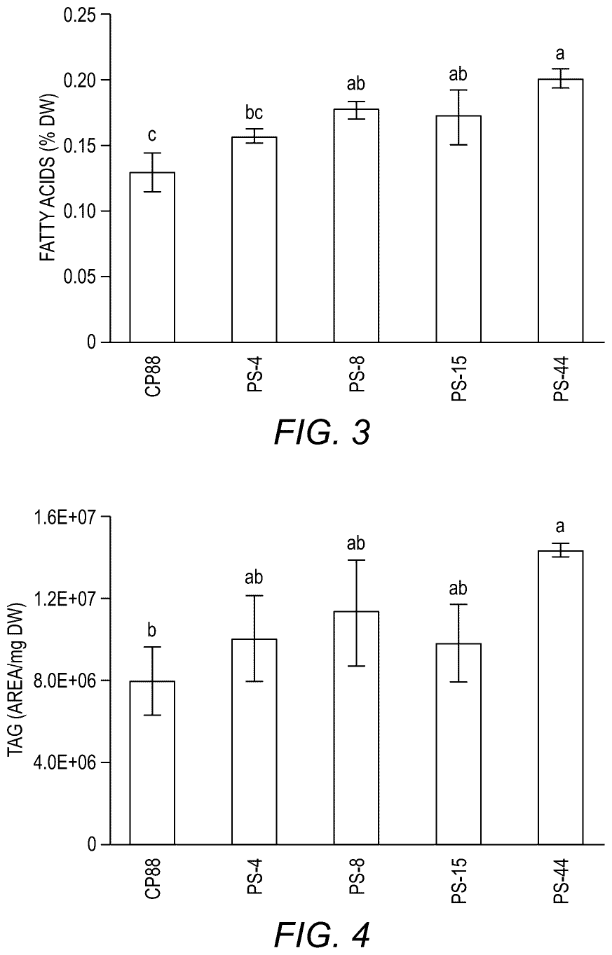

Mature internodes (IN15) were sampled for fatty acid and TAG analysis. All transgenic lines except for PS4 showed a significantly increased total fatty acid level than non-transgenic CP88-1762 control, ranging from 20.6% increase of PS-4 to 54.9% increase of PS-44 (FIG. 3). As for individual fatty acid species, palmitic acid (C16:0) and linoleic acid (C18:2) were the major components and all transgenic lines showed significant increases in linoleic acid level compared to CP88-1762 wild-type. Although a trend of increasing TAG content was observed across the PPT1 overexpressing lines, only PS44 showed a significant increase in TAG level (FIG. 4), which suggests co-engineering additional genes associated with the lipid accumulation might be necessary to achieve a substantial increase in TAG accumulation in the sugarcane stem. Pearson's correlation test among fatty acid, TAG, PPT1, and SWEET16 was performed (Table 5). The gene expression data of PPT1 and SWEET16 were multiplicative inverse transformed (shown as 1/PPT1 and 1/SWEET16) to conform to normality. Fatty acid content was positively correlated with PPT1 expression. However, no significant correlations with TAG level were observed (Table 5).

TABLE 5

| | Fatty Acid | TAG | 1/PPT1 | 1/SWEET16 |
|---|---|---|---|---|
| Fatty Acid | 1 | | | |
| TAG | 0.29 | 1 | | |
| 1/PPT1 | −0.54 | −0.37 | 1 | |
| 1/SWEET16 | −0.29 | −0.42 | 0.49 | 1 |

What is claimed is:

1. A construct for increasing fatty acid and/or oil synthesis in stem cells, comprising
(i) a first Saccharum Tonoplast Sugar Transporter (TST) promotor operably linked to nucleic acids encoding a plastidic phosphoenolpyruvate/phosphate translocator (PPT) protein; and
(ii) a second Saccharum TST promotor operably linked to nucleic acids encoding a tonoplast-localized hexose transporter protein.

2. The construct of claim 1, wherein the first Saccharum TST promoter and second Saccharum TST promoter are different.

3. The construct of claim 1, wherein the first Saccharum TST promoter and second Saccharum TST promoter comprise a nucleotide sequence selected from the group of SEQ ID NOs: 1-5.

4. The construct of claim 1, wherein the PPT protein has an amino acid sequence of SEQ ID NO: 6.

5. The construct of claim 1, wherein the tonoplast-localized hexose transporter protein has an amino acid sequence of SEQ ID NO: 7.

6. A transgenic plant or plant cell comprising the construct of claim 1.

7. The transgenic plant or plant cell of claim 6, wherein the transgenic plant or plant cell is sugarcane or energycane.

8. A method for producing a genetically modified plant or plant cell having increased fatty acid and/or oil accumulation in stem cells comprising
(a) transforming one or more host plants or plant cells with a construct of claim 1; and
(b) selecting one or more transformed plants or plant cells for increased fatty acid accumulation in stem cells as compared to a control plant or plant cell lacking the construct.

9. The method of claim 8, the method further comprising regenerating a plant from the selected plant cell comprising the construct.

10. The method of claim 8, wherein the host plant or plant cell is sugarcane or energycane.

11. A method for producing a genetically modified plant having increased fatty acid and/or oil accumulation comprising expressing an exogenous plastidic phosphoenolpyruvate/phosphate translocator (PPT) protein in a tissue of the plant thereby producing the genetically modified plant or plant cell having increased fatty acid and/or oil accumulation.

12. The method of claim 11, wherein the plant is sugarcane or energycane.

13. The method of claim 11, wherein the tissue of the plant accumulates sugar.

14. The method of claim 13, wherein the plant is sugarcane or energycane and cells of the tissue that accumulates sugar express an exogenous tonoplast-localized hexose transporter protein.

15. The method of claim 14, wherein the exogenous tonoplast-localized hexose transporter protein has an amino acid sequence of SEQ ID NO: 7.

16. The method of claim 11, wherein expression of the exogenous PPT protein is mediated by a Saccharum TST promoter comprising a nucleotide sequence selected from the group of SEQ ID NOs: 1-5.

17. The construct of claim 11, wherein the exogenous PPT protein has an amino acid sequence of SEQ ID NO: 6.
